(19) United States
(12) Patent Application Publication
(10) Pub. No.: US 2006/0165668 A1
(43) Pub. Date: Jul. 27, 2006

(54) Genetically modified tumor cells as cancer vaccines

(76) Inventors: Linda N. Liu, Clarksville, MD (US); Jonathan M. Weiss, Rockville, MD (US); LinHong Li, North Potomac, MD (US)

Correspondence Address: FULBRIGHT & JAWORSKI L.L.P., 600 CONGRESS AVE., SUITE 2400, AUSTIN, TX, 78701, US

(21) Appl. No.: 11/298,311

(22) Filed: Dec. 9, 2005

Related U.S. Application Data
(60) Provisional application No. 60/634,919, filed on Dec. 10, 2004.

(51) Int. Cl. A61K 48/00; C07H 21/04; C12P 21/04; C12N 5/08; C12N 15/87

(52) U.S. Cl. 424/93.21; 435/69.52; 435/459; 435/366; 536/23.5

(57) ABSTRACT

The present invention provides methods and compositions for electroporation-mediated gene transfer to cancer cells. The transfected cancer cells are genetically modified to express one or more therapeutic proteins. In certain embodiments, the cancer cells are modified to express one or more cytokines capable of enhancing the immunogenicity of the transfected cancer cell. Administering the transfected cancer cell to a subject will lead to enhanced immune-cell mediated killing of tumors. Accordingly, the present invention provides methods and compositions for improved treatment and prevention of cancer and other hyperproliferative diseases.

This application claims priority to U.S. Provisional Application No. 60/634,919 filed Dec. 10, 2004, which is incorporated herein in its entirety.

### BACKGROUND OF THE INVENTION

1. Field of the Invention

The present invention relates generally to the fields of cell biology, cancer biology, and immunology. More particularly, it concerns genetically modified tumor cells for use in the treatment of cancer.

2. Description of Related Art

In spite of recent medical advances, cancer continues to represent a significant national and worldwide health problem. One strategy used to combat this disease involves immunotherapy, whereby the body's own immune system is used to destroy cancerous cells. As a result, immunotherapy as a means to treat or prevent cancer is the subject of considerable research interest.

Immune response to tumor-associated antigens (TAA) typically begins with uptake of the antigen by antigen presenting cells (APCs), such as dendritic cells (DCs), macrophages, and B cells. APCs present the antigens to naïve and memory T cells (Van Schooten et al., 1997; Mellman and Steinman, 2001).

Several studies have explored the possibility of using gene-modified tumor cells to enhance tumor immunogenicity and consequent host recognition and elimination of tumor cells. However, these studies have primarily utilized viral vectors for gene transfer. For example, transfection of tumor cells with retrovirus vectors containing the IL-2 gene have been reported (Karp et al., 1993; Gansbacher et al., 1990; Connor et al., 1993). Studies using tumor cells transduced with herpes simplex virus (HSV) vector containing IL-2 have also been reported (Kimura et al., 2003). Kimura et al. also described studies using tumor cells transduced with a retrovirus vector containing IL-12. Retrovirally transduced tumor cells secreting IL-21 have been shown to generate antitumor responses in mice (Ma et al., 2003). In addition, transduction of tumor cells with retrovirus vectors containing the GM-CSF gene have been reported (Dranoff et al., 1993; Jain et al., 2003).

These viral-vector mediated approaches show the potential for using genetically modified tumor cells as a cancer vaccine. However, safer, more effective, and more efficient methods of genetically modifying cancer cells are needed for the development of cancer vaccines.

Modification of tumor cells by electroporation would avoid virus-related risks and reduce labor and time. Electroporation has been described as a means to introduce nonpermeant molecules into living cells (reviewed in Mir, 2000). Electroporation is most commonly used to introduce DNA (Knutson and Yee, 1987) and RNA (Van Meirvenne et al., 2002; Van Tendeloo et al., 2001) into cells, but it has also been described as a means of introducing other macromolecules into the cytoplasm of living cells (Zhou et al., 1995; Harding, 1992; Chen et al., 1993; Li et al., 1994; Kim et al., 2002). Nevertheless, methods are lacking for efficient use of electroporation in the treatment of cancer and other hyperproliferative diseases. Development of such techniques would represent a significant advance in cancer therapeutics.

### SUMMARY OF THE INVENTION

The present invention provides methods and compositions for electroporation-mediated gene transfer to cancer cells and other hyperproliferative cells. These methods and compositions provide improved treatments for cancer and other hyperproliferative diseases.

In one embodiment, the present invention provides a method of producing a cancer cell expressing a therapeutic protein, the method comprising: obtaining a cancer cell composition; and transfecting cancer cells of the composition by electroporation with one or more nucleic acid molecules encoding one or more therapeutic proteins; wherein, the transfected cancer cells express one or more therapeutic proteins.

In another embodiment, the present invention provides a method of treating cancer in a subject comprising: obtaining a cancer cell composition; transfecting cancer cells of the composition by electroporation with one or more nucleic acid molecules encoding one or more therapeutic proteins; and administering the transfected cells to the subject.

In certain embodiments, the present invention provides a method of treating cancer in a subject comprising: obtaining a cancer cell composition; dividing the cancer cell composition in to a first cancer cell composition and a second cancer cell composition; transfecting cancer cells of the first cancer cell composition by electroporation with one or more nucleic acid molecules encoding one or more therapeutic proteins; transfecting cancer cells of the second cancer cell composition by electroporation with one or more nucleic acid molecules encoding one or more therapeutic proteins, wherein the one or more nucleic acid molecules introduced into the cancer cells of the second cancer cell composition are different from the one or more nucleic acid molecules introduced into the cancer cells of the first cancer cell composition; and administering the transfected cancer cells of the first cancer cell composition and the second cancer cell composition to the subject. In one embodiment, the therapeutic protein in the first cancer cell composition is IL-2 and the therapeutic protein in the second cancer cell composition is CD40L.

In yet another embodiment, the present invention provides a method for eliciting an immune response to a cancer cell in a subject comprising: obtaining a cancer cell composition; transfecting cancer cells of the composition by electroporation with one or more nucleic acid molecules encoding one or more immuno-stimulatory proteins; and administering the transfected cells to the subject.

A “cancer cell composition” may be any composition comprising a cancer cell. Any cancer cell composition may be used in the practice of the present invention. For example, the cancer cell composition may be obtained from a culture, tissue, organ or organism. In certain embodiments, the cancer cell composition may comprise autologous cancer cells from the subject being treated. In other embodiments, the cancer cell composition comprises allogenic cancer cells. Those of skill in the art are familiar with methods for obtaining cancer cells from a subject. For example, the cancer cell composition may be obtained by biopsy, aspiration, surgical resection, venipuncture, or leukapheresis. In certain aspects, the cancer cell composition is expanded in culture prior to transfection.

Although cells of any cancer type are contemplated by the present invention, particular examples of cancer cells include breast cancer cells, lung cancer cells, prostate cancer cells, ovarian cancer cells, brain cancer cells, liver cancer cells, cervical cancer cells, colon cancer cells, renal cancer cells, skin cancer cells, head & neck cancer cells, bone cancer cells, esophageal cancer cells, bladder cancer cells, uterine cancer cells, lymphatic cancer cells, stomach cancer cells, pancreatic cancer cells, testicular cancer cells, or leukemia cells.

In certain embodiments, the subject is an animal. More preferably, the animal is a mammal. In certain embodiments, the mammal is a mouse or a rat. In a preferred embodiment, the mammal is a human. In certain aspects, the subject has a hyperproliferative disease such as cancer. In other aspects, the subject is at risk for developing cancer.

A “therapeutic protein” is a protein that can be administered to a subject for the purpose of treating or preventing a disease. For example, a therapeutic protein can be a protein administered to a subject for treatment or prevention of cancer. Examples of classes of therapeutic proteins include tumor suppressors, inducers of apoptosis, cell cycle regulators, immuno-stimulatory proteins, toxins, cytokines, enzymes, antibodies, inhibitors of angiogenesis, metalloproteinase inhibitors, hormones or peptide hormones.

An “immuno-stimulatory protein” is a protein involved in the activation, chemotaxis, or differentiation of immune cells. Examples of classes of immuno-stimulatory proteins include thymic hormones, cytokines, and growth factors as well as their respective receptors or ligands.

Thymic hormones include, for example, prothymosin-α, thymulin, thymic humoral factor (THF), THF-γ-2, thymocyte growth peptide (TGP), thymopoietin (TPO), thymopentin, and thymosin-α-1.

In certain embodiments, the cancer cell is transfected with nucleic acid molecules encoding at least 1, 2, 3, 4, 5, or more therapeutic proteins. When a cancer cell is modified to express two or more therapeutic proteins, the therapeutic proteins may be encoded by the same nucleic acid molecule or they may be encoded by separate nucleic acid molecules.

In certain embodiments, the cancer cell is transfected with nucleic acid molecules encoding at least 1, 2, 3, 4, 5, or more immuno-stimulatory proteins. When a cancer cell is modified to express two or more immuno-stimulatory proteins, the immuno-stimulatory proteins may be encoded by the same nucleic acid molecule or they may be encoded by separate nucleic acid molecules.

In a preferred embodiment, the cancer cells are transfected with a nucleic acid molecule encoding a cytokine. It is contemplated that the cytokine may be derived from any species. In particular embodiments, the cytokines are human or murine. In certain aspects, the cancer cells are transfected with one or more nucleic acid molecules encoding at least 2, 3, 4, 5, or more cytokines.

Examples of cytokines include, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, leukocyte inhibitory factor (LIF), IFN-α, IFN-β, IFN-γ, TNF, TNF-α, TGF-β, G-CSF, M-CSF, and GM-CSF.

In certain embodiments, the cytokines are pro-inflammatory cytokines such as IL-1α, IL-1β, IL-2, IL-6, IL-8, TNF-α, leukocyte inhibitory factor (LIEF), IFN-γ, GM-CSF, M-CSF, IL-11, IL-12, IL-15, IL-17, and IL-18.

As mentioned above, the cancer cells may be transfected with nucleic acid molecules encoding more than one cytokine. In one embodiment, the cancer cells are transfected with IL-15 and IL-21. In some embodiments, the cancer cells are transfected with IL-15, IL-21, and GM-CSF. In another embodiment, the cancer cells are transfected with IL-12, IL-21, and GM-CSF. In yet another embodiment, the cancer cells are transfected with IL-12, IL-15, and IL-21. In some embodiments, the cancer cells are transfected with IL-12, IL-15, and GM-CSF. In other embodiments, the cancer cells are transfected with IL-15 and IL-18. In certain embodiments, the cancer cells are transfected with IL-12 and IL-18. In some embodiments, the cancer cells are transfected with IL-18 and IL-21.

Other immuno-stimulatory proteins include B7.1 (CD80), B7.2 (CD86), ICAM-1 (CD54), VCAM-1, LFA-1, VLA-4, CD40, CD40L (CD154), Flt-3, Flt-3L, 4-1BBL, CD27, CD28, CD32, CD40, CD70, CD83, CD154, MHC class I, and MHC class II. In certain aspects of the invention, the cancer cells are transfected with a nucleic acid molecule encoding one or more of B7.1 (CD80), B7.2 (CD86), ICAM-1 (CD54), VCAM-1, LFA-1, VLA-4, CD40, and CD40L. In some embodiments, the cancer cells are also transfected with nucleic acid molecules encoding one or more cytokines. For example, the cancer cells may be transfected with nucleic acid molecules encoding CD40L and IL-2. In some embodiments, the cancer cells are transfected with CD40, CD40L, and IL-2. In other embodiments, the cancer cells are transfected with CD40, CD40L, and IL-18. In certain embodiments, the cancer cells are transfected with CD40, CD40L, and IL-12. In some embodiments, the cancer cells are transfected with nucleic acid molecules encoding ICAM-1 and LFA-1. In other embodiments, the cancer cells are transfected with nucleic acid molecules encoding VCAM-1 and VLA-4.

In one embodiment, the nucleic acid molecules is a DNA. In another embodiment, the nucleic acid molecule is an RNA.

Co-stimulatory or inhibitory signaling receptors and ligands are involved in determining the activation, expansion, and effector functions of immune cells, and ultimately the final targeting and execution of immune functions. One mechanism by which cancer cells may evade the immune system is through the expression of immune inhibitory molecules. Accordingly, it is contemplated that in certain aspects of the invention the cancer cell may be transfected with 1, 2, 3, 4, or more different nucleic acid sequences that can inhibit or interfere with the expression of target genes. Examples of such nucleic acid sequences include aptamers, ribosomal RNA, splicosomal RNA, antisense RNA, dsRNA, siRNA, and/or miRNA. For example, the introduction of inhibitory RNA molecules into a cell can result in post-transcriptional gene silencing (PTGS). In PTGS, the transcript of the silenced gene is synthesized but protein does not accumulate because the transcript is rapidly degraded or because translation of the transcript is inhibited. In certain aspects of the present invention, the targeted gene or genes may include genes that inhibit immune response or genes that regulate the expression of genes that inhibit immune response. Examples of genes that may inhibit immune response include, TGF-β, CD200, CD200R. HLA-E, HLA-G, and Toll-like receptors (e.g., TLR4). It should also be noted that immunomodulatory molecules, such as cytokines, may have immuno-stimulatory or immuno-inhibitory properties depending on the circumstances (e.g. growth state of the cells, the type of neighboring cells, cytokine concentrations, the combination of other cytokines present at the same time, and/or the temporal sequence of several cytokines acting on the same cell). Thus, it may be desirable to inhibit certain cytokines in the transfected cancer cell while overexpressing others. It is further contemplated that in certain aspects of the invention, a cancer cell is transfected with one or more nucleic acid sequences that inhibit or interfere with the expression of a target gene and one or more nucleic acid sequences encoding one or more therapeutic proteins.

Those of skill in the art are familiar with methods of electroporation. The electroporation may be, for example, flow electroporation or static electroporation. In one embodiment, the method of transfecting the cancer cells comprises use of an electroporation device as described in U.S. patent application Ser. No. 10/225,446, incorporated herein by reference. Methods and devices for electroporation are also described in, for example, published PCT Application Nos. WO 03/018751 and WO 2004/031353; U.S. patent application Ser. Nos. 10/781,440, 10/080,272, and 10/675,592; and U.S. Pat. Nos. 5,720,921, 6,074,605, 6,773,669, 6,090,617, 6,485,961, 6,617,154, 5,612,207, all of which are incorporated by reference.

The transfected cancer cells of the present invention can be administered to a subject by methods well known to those of skill in the art. For example, the transfected cancer cells may be administered by intravenous injection, intramuscular injection, intratumoral injection, subcutaneous injection, or leukapheresis. It is also contemplated that the transfected cancer cells may be administered intranodally, intralymphaticly, or intraperitoneally. The transfected cancer cells may be administered to the subject at or near a tumor in the subject, or to a site from which a tumor has been surgically removed from the subject. However, it is not necessary that the transfected cancer cells be administered at the tumor site to achieve a therapeutic effect. Thus, in certain embodiments the transfected cancer cells may be administered at a site distant from the tumor site. Those of skill in the art will be able to determine the best method for administering the transfected cancer cells to an individual subject.

It is desirable to inactivate the transfected cancer cells prior to administering them to the subject. Those of skill in the art are familiar with methods for inactivating cells. In some embodiments, the transfected cancer cells are inactivated by a cytostatic agent or a cytotoxic agent. In other embodiments, transfected cancer cells are inactivated by irradiation. In another embodiment, the transfected cancer cells are co-transfected with a suicide gene, such as HSV-TK. A cancer cell transfected with HSV-TK could then be killed after it was administered to the subject by giving the subject ganciclovir. A combination of cell inactivating methods may also be used.

In addition to being transfected with nucleic acid molecules encoding therapeutic proteins, the cancer cells may also be transfected with marker genes. A marker gene encodes a protein that facilitates the detection of the transfected cancer cell.

In particular embodiments, cancer cells transfected with one or more nucleic acid molecules encoding one or more immuno-stimulatory proteins are administered to the subject as a vaccine. The vaccine may be used therapeutically or preventatively. A therapeutic vaccine is administered to a subject having cancer to treat the cancer. In a subject having cancer, the vaccine may be made from the subject's own cancer cells. However, allogenic cancer cells could also be used. A preventative vaccine is administered to a subject without cancer to reduce the risk of the subject developing cancer.

In one embodiment, the present invention provides a method of treating cancer in a subject comprising: obtaining a cancer cell from the subject; transfecting the cancer cell with one or more nucleic acid molecules encoding three cytokines; and administering the transfected cells to the subject. In certain embodiments, the three cytokines are further defined as IL-15/IL-21/GM-CSF, IL-12/IL-21/GM-CSF, IL-12/IL-15/IL-21, or IL-12/IL-15/GM-CSF. In certain aspects, the method further comprises inactivating the transfected cell prior to administering the transfected cell to the subject.

In other embodiments, the present invention provides a vaccine comprising a cancer cell genetically modified to over express two cytokines as compared to an unmodified cancer cell. In some embodiments the cancer cell is an autologous cancer cell derived from the subject to be treated with the vaccine. In other embodiments the cancer cell is an allogenic cancer cell. In some aspects, the cancer cell is inactivated. In certain embodiments, the two cytokines are further defined as IL-15 and IL-21. In some aspects, the vaccine further comprises a pharmaceutically acceptable carrier.

In yet other embodiments, the present invention provides a vaccine comprising a cancer cell genetically modified to overexpress three cytokines as compared to an unmodified cancer cell. In some embodiments the cancer cell is an autologous cancer cell derived from the subject to be treated with the vaccine. In other embodiments the cancer cell is an allogenic cancer cell. In some aspects, the cancer cell is inactivated. In certain embodiments, the three cytokines are further defined as IL-15/IL-21/GM-CSF, IL-12/IL-21/GM-CSF, IL-12/IL-15/IL-21, or IL-12/IL-15/GM-CSF. In some aspects, the vaccine further comprises a pharmaceutically acceptable carrier.

In some embodiments, the present invention provides a vaccine comprising a cancer cell genetically modified to over express CD40L and one or more cytokines as compared to an unmodified cancer cell. In some embodiments the cancer cell is an autologous cancer cell derived from the subject to be treated with the vaccine. In other embodiments the cancer cell is an allogenic cancer cell. In certain aspects of the invention the cancer cell is a leukemia cell, such as a B cell from a patient with chronic lymphocytic leukemia or acute lymphocytic leukemia. In some aspects, the cancer cell is inactivated. In certain embodiments, the vaccine comprises a cancer cell genetically modified to over express CD40L and IL-2. In some aspects, the vaccine further comprises a pharmaceutically acceptable carrier.

The present invention also provides methods for activating and expanding B cells ex vivo. These methods will be useful for activating and expanding B cells for a variety of applications including, for example, cellular therapy, immunotherapy, drug screening, or antigen screening.

In some embodiments, the present invention provides a method for activating a B cell ex vivo comprising: providing a first B cell; providing a second B cell that is genetically modified to overexpress CD40L; and culturing said first B cell in the presence of said second B cell, wherein said first B cell is activated. In another embodiment, the present invention provides a method for activating a B cell ex vivo comprising: providing a first B cell; providing a second B cell; electroporating said second B cell with a nucleic acid that encodes CD40L; and culturing said first B cell in the presence of said second B cell, wherein said first B cell is activated. In other embodiments, the present invention provides a method for activating a B cell ex vivo comprising: providing a B cell; providing a peripheral blood mononuclear cell (PBMC); electroporating said PBMC with a nucleic acid that encodes CD40L; and culturing said B cell in the presence of said PBMC, wherein said B cell is activated. In certain embodiments of the invention, the method further comprises expanding the activated B cell ex vivo.

The first B cell and the second B cell may be autologous or allogenic. In certain embodiments, the first B cell is a leukemia cell (e.g., a B-CLL cell). In some embodiments, the second B cell is a leukemia cell. In some embodiments, both the first B cell and the second B cell are leukemia cells. It is contemplated that the ability to activate and expand leukemic B cells can be used in combination with the methods of treating cancer described herein.

In some embodiments, the present invention provides a method for activating B cells ex vivo comprising: obtaining a first and a second population of B cells; electroporating said second population of B cells to introduce a nucleic acid encoding CD40L; and culturing said first population of B cells in the presence of said second population of B cells, wherein said first population of B cells is activated.

In certain embodiments, the present invention provides a method for activating B cells ex vivo comprising: obtaining a population of B cells; dividing said population of B cells into a first population and a second population; electroporating said second population of B cells to introduce a nucleic acid encoding CD40L; and culturing said first population of B cells in the presence of said second population of B cells, wherein said first population of B cells is activated. In certain embodiments of the invention, the method further comprises expanding the activated first population of B cells ex vivo. In some embodiments, the method further comprises freezing one or more aliquots of the transfected B cells for storage. The frozen cells could then be thawed as needed and co-cultured with untransfected B cells to activate the untransfected B cells.

In certain aspects of the invention, the population of B cells is obtained from a subject. The cells may be obtained by any method known in the art. In a preferred embodiment, the cells are obtained from the peripheral blood of the subject. In certain embodiments, the subject has leukemia.

As used herein, “activation” or “activating” refers to the stimulation of a B cell to proliferate and/or differentiate. Thus, an “activated B cell” refers to a B cell that has been signaled to proliferate and/or differentiate. This is in contrast to a naïve B cell, which is typically quiescent. Those of skill in the art will be familiar with methods of identifying an activated B cell. One method is to simply observe the proliferation of the activated B cells. Other approaches include assessing the expression of one or more molecules, such as co-stimulatory molecules (e.g., CD80, CD86) or adhesion molecules (e.g., ICAM-I), that are upregulated in activated B cells.

A B cell or a PBMC can be genetically modified to overexpress CD40L by using electroporation to transfect the cell with a nucleic acid encoding CD40L. The electroporation may be flow electroporation or static electroporation. In one embodiment, the method of transfecting the cell comprises use of an electroporation device as described in published PCT Application No. WO 03/018751, which is incorporated by reference. Methods and devices for electroporation that may be used in the context of the present invention are also described in, for example, published PCT Application Nos. WO 03/018751 and WO 2004/031353; U.S. patent application Ser. Nos. 10/225,446, 10/781,440, 10/080,272, and 10/675,592; and U.S. Pat. Nos. 5,720,921, 6,074,605, 6,773,669, 6,090,617, 6,485,961, 6,617,154, 5,612,207, all of which are incorporated by reference.

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. RENCA tumor cells modified to co-express three cytokines (IL-12/IL-21/GM-CSF, IL-12/IL-15/IL-21, IL-12/IL-15/GM-CSF, or IL-15/IL-21/GM-CSF) were evaluated for their ability to inhibit established tumor cell growth. Balb/C mice were injected with 5e5 RENCA cells at day 0. When primary tumors were established (day 7), the mice were injected subcutaneously with 5e5 non-viral, genetically modified RENCA cells at a remote site. The control group received RENCA cells that were electroporated without DNA. The established primary tumors were measured every 4 days. All modified RENCA cells slowed the growth of the primary tumor to some degree relative to controls. The greatest effect was observed with the IL-15/IL-21/GM-CSF modified RENCA cells, which inhibited primary tumor growth significantly at all time points.

FIG. 2. Weighing of the primary tumors removed at day 26 revealed significantly smaller tumors from mice receiving IL-15/IL-21/GM-CSF modified RENCA cell treatment relative to the control group (p<0.02).

FIG. 3. RENCA tumor cells modified to express IL-15, IL-21, and GM-CSF, alone or in various combinations, were evaluated for their ability to inhibit established tumor cell growth. Balb/C mice were injected with 5e5 RENCA cells at day 0. When primary tumors were established (day 7), the mice were injected subcutaneously with 5e5 non-viral, genetically modified RENCA cells at a remote site. The control group received either RENCA cells electroporated without DNA or RENCA cells electroporated with an empty vector. The established primary tumors were measured two times per week for a total of three weeks. FIG. 3 is a graph of the primary tumor area in mice injected with genetically modified RENCA cells expressing IL-15/IL-21 or IL-15/IL-21/GM-CSF. RENCA cells transfected with an empty vector were used as a control. The graph shows that co-expression of IL-15 and IL-21, either with or without GM-CSF, elicited a significant reduction in tumor area.

FIG. 4. Cryopreserved primary B-CLL cells were thawed and then either immediately (0 min) transfected or were incubated at 37° C. for 5, 30 or 60 minutes prior to hCD40L DNA transfection. The hCD40L-transfected B-CLL cells were analyzed by FACS at 3 hours post transfection after the cells were immunostained with FITC-conjugated Mab against hCD40L and stained with PI. Without post-thawing incubation, hCD40L-transfected B-CLL cell viability was as low as 48%, and among the viable cells only 11% expressed hCD40L. In contrast, if the thawed cells were allowed 30 min incubation prior to electroporation, the hCD40L-transfected B-CLL cell viability reached 80%, and approximately 40% of the viable cells expressed hCD40L. No significant improvement was observed with incubation times longer than 30 minutes.

FIG. 5A and FIG. 5B. FIGS. 5A and 5B show cell viability and hCD40L expression of cryopreserved hCD40L-transfected B-CLL cells during long-term tissue culture. Seven CLL patients' samples (donors #1 to #7) were analyzed. The hCD40L-transfected B-CLL cells were cryopreserved at 3 hours post transfection and stored in liquid nitrogen. The cryopreserved hCD40L-transfected cells were thawed and either immediately analyzed for hCD40L expression and cell viability (donors #1, #2, and #3) or irradiated with 30Gy γ-radiation (donors #4 to #7) before analysis. The cells were also cultured at 37° C. in a CO₂ incubator for 24 and 48 hours. Decreased cell viability was observed across all patients' samples (FIG. 5A). However, hCD40L expression maintained at a range from 40% to 90% of the viable cells up to 48 hrs (FIG. 5B).

FIG. 6. FIG. 6 shows the stability of cryopreserved hCD40L-transfected B-CLL cells. The hCD40L-transfected B-CLL cells were cryopreserved at 3 hours post transfection and stored in liquid nitrogen up to 5 months (Donors #4 to #7 ) and 8 months (Donor #4). The cryopreserved cells were thawed at the indicated time points and analyzed for the hCD40L transgene expression and cell viability, which were compared to the results prior to cryopreservation. No significant alteration of the cell viability and the transgene hCD40L expression was observed on the thawed hCD40L-transfected B-CLL cells up to 5 months (for donors #4 to #7) and 8 months (donor #4).

FIG. 7A and FIG. 7B. FIGS. 7A and 7B demonstrate the upregulation of accessory molecules in hCD40L-transfected B-CLL cells. Primary B-CLL cells were transfected with hCD40L DNA plasmid and immunostained with FITC-conjugated Mab against hCD40L, HLA-DR, CD86, CD80 and CD54 (ICAM-1) and analyzed by FACS 48 hours post transfection. Upregulation of accessory molecules was observed in various hCD40L-transfected B-CLL patients samples. Data from 3 donors are summarized in FIGS. 7a and 7B. The percentage of CD80 expression cells was significantly increased (p<0.04) (FIG. 7A), though the expression level as indicated by the mean fluorescence intensity did not increase much (FIG. 7B). The other 3 accessory protein expression levels examined improved remarkably after hCD40L transfection (FIG. 7B), though escalation of the positive cell population was moderate (FIG. 7A).

FIG. 8. hCD40L-transfected B-CLL cells elicited IFN-γ secretion in a mixed lymphocyte reaction. In a well on a 96-well plate, 4e5 allogeneic lymphocytes were mixed with 2e5 hIL2-transfected B-CLL cells and 4e5 hCD40L-transfected B-CLL cells (black bar) or control B-CLL cells (empty bar) and cocultured for 40 to 48 hours. The conditioned culture media was analyzed for IFN-γ production by a commercially available ELISA kit (R&D System). The standard deviation was given from 4 repeated experiments. The p value of the student t-test was p<0.001.

FIG. 9. As shown in FIG. 9, sustained hCD40L expression was observed in hCD40L-transfected CLL-B cells. PBMCs were transfected by electroporation with mRNA encoding for CD40L. The expression of the hCD40L was monitored up to 72 hours post transfection. No hCD40L expression was observed on the control CLL-B cells. Approximately, 60% of the transfected CLL-B cells expressed hCD40L when analyzed by FACS at 2-4 hours post transfection; it then decreased to about 30% and was sustained up to 72 hours.

FIG. 10. Transfection of mRNA did not affect CLL-B cell viability. Viability of control and transfected CLL-B cells was monitored up to 72 hours post mRNA transfection. Viability of control CLL-B cells decreased from 90% to 50% when they were under normal tissue culture condition. There was no significant increase of non-viable cells of mRNA transfected CLL-B cells under the same culture conditions.

FIGS. 11A, 11B, 11C, and 11D. Forced expression of hCD40L upregulates immuno-accessory molecule expression in CLL-B cells. PBMCs were transfected with mRNA encoding for hCD40L. Cells were analyzed for CD86 (FIG. 11A), CD80 (FIG. 11B), CD54 (FIG. 11C), and HLA-DR (FIG. 11D) expression by FACS at 24, 48, and 72 hours post transfection (except CD80) as indicated in FIGS. 11A-11D. The mean fluorescence intensity of each molecule was monitored up to 72 hours. As shown in FIGS. 11A-11D, significant increase of CD86, CD80, CD54, and HLA-DR was observed.

FIG. 12. hCD40L expressing CLL cells elicit allo-T cells response. Control and transfected CLL-B cells were mixed and co-cultured with allo-lymphocytes 1-3 hours post transfection. IFN-γ production was measured with a commercially available ELISA kit after co-culture for 3 days. The transfected cells elicited a significantly higher level of IFN-γ production than control cells (p<0.002, student t-test).

### DESCRIPTION OF ILLUSTRATIVE EMBODIMENTS

The present invention provides methods and compositions for the prevention and treatment of cancer and other hyperproliferative diseases. For example, in one embodiment the present invention provides a method of treating cancer in a subject comprising: obtaining a cancer cell composition; transfecting cancer cells of the composition by electroporation with one or more nucleic acid molecules encoding one or more therapeutic proteins; and administering the transfected cells to the subject.

The modification of cancer cells according the methods of the present invention will provide a cancer vaccine with enhanced potency. For example, a cancer cell could be modified with a combination of IL-15 and IL-21, or a combination of IL-15, IL-21, and GM-CSF, or a combination of IL-2 and CD40L. The modified cancer cell would then be inactivated and administered to the subject.

IL-21, a recently characterized T cell derived cytokine that regulates natural killer (NK) and T cell function, has been shown as a key factor in the transition between innate and adaptive immune response. IL-15 in synergy with IL-21 enhances IFN-γ production in human NK and T cells. Thus, IL-21/IL-15 modified cancer cells would promote NK and T cell activity. GM-CSF is known to attract dendritic cells (DCs). Thus, if the IL-21/IL-15 modified cancer cells are also genetically modified with GM-CSF, they will also attract DCs. The DCs will phagocytize the cancer cells and process/present them to the nearby, active NK and T cells. Thus, by combining cytokines that act on multiple cell types, a more effective immune response will be developed by the host. With this approach, it does not matter if the tumor antigen(s) are unknown, since a wide range of tumor cell antigens would be expected to be exposed and presented to killer cells.

The genetically modified tumor cell could be either an autologous cell or an allogenic cell. If a patient's own tumor cells could be collected, they would be attractive cells for gene modification. Once transfected with therapeutic genes, they can be irradiated, or otherwise inactivated, and reintroduced back into the same patient, with minimized concern.

### A. HYPERPROLIFERATIVE DISEASES

The invention may be used in the treatment and prevention of hyperproliferative diseases including, but not limited to, cancer. A hyperproliferative disease is any disease or condition which has, as part of its pathology, an abnormal increase in cell number. Included in such diseases are benign conditions such as benign prostatic hypertrophy and ovarian cysts. Also included are premalignant lesions, such as squamous hyperplasia. At the other end of the spectrum of hyperproliferative diseases are cancers. A hyperproliferative disease can involve cells of any cell type. The hyperproliferative disease may or may not be associated with an increase in size of individual cells compared to normal cells.

Another type of hyperproliferative disease is a hyperproliferative lesion, a lesion characterized by an abnormal increase in the number of cells. This increase in the number of cells may or may not be associated with an increase in size of the lesion. Examples of hyperproliferative lesions that are contemplated for treatment include benign tumors and premalignant lesions. Examples include, but are not limited to, squamous cell hyperplastic lesions, premalignant epithelial lesions, psoriatic lesions, cutaneous warts, periungual warts, anogenital warts, epidermdysplasia verruciformis, intraepithelial neoplastic lesions, focal epithelial hyperplasia, conjunctival papilloma, conjunctival carcinoma, or squamous carcinoma lesion. The lesion can involve cells of any cell type. Examples include keratinocytes, epithelial cells, skin cells, and mucosal cells.

### B. CANCER

The present invention provides methods and compositions for the treatment and prevention of cancer. Cancer is one of the leading causes of death, being responsible for approximately 526,000 deaths in the United States each year. The term “cancer” as used herein is defined as a tissue of uncontrolled growth or proliferation of cells, such as a tumor.

Cancer develops through the accumulation of genetic alterations (Fearon and Vogelstein, 1990) and gains a growth advantage over normal surrounding cells. The genetic transformation of normal cells to neoplastic cells occurs through a series of progressive steps. Genetic progression models have been studied in some cancers, such as head and neck cancer (Califano et al., 1996). Treatment and prevention of any type of cancer is contemplated by the present invention. The present invention also contemplates methods of prevention of cancer in a subject with a history of cancer. Examples of cancers have been listed above.

### C. ELECTROPORATION

Certain embodiments involve the use of electroporation to facilitate the entry of one or more nucleic acid molecules encoding one or more therapeutic proteins into a cancer cell. Any cancer cell is contemplated by the present invention. The cancer cell may be an autologous cancer cell or an allogenic cancer cell.

As used herein, “electroporation” refers to application of an electrical current or electrical field to a cell to facilitate entry of a nucleic acid molecule into the cell. One of skill in the art would understand that any method and technique of electroporation is contemplated by the present invention. However, in certain embodiments of the invention, electroporation may be carried out as described in U.S. Patent application Ser. No. 10/225,446, filed Aug. 21, 2002, the entire disclosure of which is specifically incorporated herein by reference.

In other embodiments of the invention, electroporation may be carried out as described in U.S. Pat. No. 5,612,207 (specifically incorporated herein by reference), U.S. Pat. No. 5,720,921 (specifically incorporated herein by reference), U.S. Pat. No. 6,074,605 (specifically incorporated herein by reference); U.S. Pat. No. 6,090,617 (specifically incorporated herein by reference); and U.S. Pat. No. 6,485,961 (specifically incorporated herein by reference).

Other methods and devices for electroporation that may be used in the context of the present invention are also described in, for example, published PCT Application Nos. WO 03/018751 and WO 2004/031353; U.S. patent application Ser. Nos. 10/781,440, 10/080,272, and 10/675,592; and U.S. Pat. Nos. 6,773,669, 6,090,617, 6,617,154, all of which are incorporated by reference.

Electroporation has been described as a means to introduce nonpermeant molecules into living cells (reviewed in Mir, 2000). At the level of the entire cell, the consequences of cell exposure to the electric pulses are not completely understood. In the presence of the external electric field, a change in the transmembrane potential difference is believed to be generated (Neumann et al., 1999; Weaver and Chizmadzhev, 1996; Kakorin et al., 1996). It superimposes upon the resting transmembrane potential difference and it may be calculated from the Maxwell's equations, providing a few approximations are made (very reduced thickness of the cell membrane, null membrane conductivity, etc.) (Mir, 2000). These changes in the transmembrane potential difference have been experimentally observed (Hibino et al., 1993; Gabriel and Teissié, 1999). Analytically, the effects of the exposure of cells to electric pulses are well described in the case of isolated cells in suspension (Kotnik et al., 1998).

At the molecular level of analysis, the explanation of the phenomena occurring at the cell membrane level is hypothetical. It is assumed that above a threshold value of the net transmembrane potential, the changes occurring in membrane structure will be enough as to render that membrane permeable to otherwise nonpermeant molecules of given physicochemical characteristics (molecular mass, radius, etc.) (Mir, 2000).

DNA electroporation was originally described using simple generators that produce exponentially decaying pulses. Square-wave electric pulse generators were later developed that allowed specification of the various electric parameters (pulse intensity, pulse length, number of pulses) (Rols and Teissié, 1990). The selection of parameters is dependent on the cell type being electroporated and physical characteristics of the molecules that are to be taken up by the cell.

The inventors have demonstrated previously the efficient electroporation-mediated genetic modification of many types of cancer cells including: B16 murine melanoma (Weiss et al. 2003); RENCA (Weiss et al., 2003); and CLL-B cells (Li et al., 2002).

### D. CELLULAR VACCINES

In certain embodiments, the present invention provides methods and compositions for eliciting an immune response to a cancer cell in a subject. For example, cancer cells transfected with one or more nucleic acid molecules encoding one or more therapeutic proteins may be administered to a subject as a cellular vaccine. A “cellular vaccine” or “whole cell vaccine” is a vaccine made from whole cancer cells. The vaccine may be preventative or therapeutic. A preventative vaccination is given prior to the subject developing a disease. A therapeutic vaccination is given to a subject who already has the disease.

Any cancer cell may be used in the practice of the present invention. The cancer cells may be isolated from a culture, tissue, organ or organism. In certain embodiments, the cancer cells may be isolated from the subject that is to be vaccinated (i.e., an autologous cellular vaccine). Techniques that are well-known to those of skill in the art may be used to isolate the cancer cells from a subject. For example, the cancer cells may be isolated by biopsy, aspiration, surgical resection, venipuncture, or leukapheresis. In certain aspects, the cancer cells are expanded in culture prior to transfection.

In particular embodiments, cancer cells transfected with one or more nucleic acid molecules encoding one or more immuno-stimulatory proteins are administered to the subject as a vaccine. In addition to being transfected with nucleic acid molecules encoding therapeutic proteins, the cancer cells may also be transfected with marker genes. A marker gene encodes a protein that facilitates the detection of the transfected cancer cell.

It is also desirable to inactivate the transfected cancer cells before administering them to a subject. Those of skill in the art are familiar with several methods for inactivating cells. Any method may be used as long as it allows the cells to express the therapeutic protein while preventing the cells from proliferating. A common approach to inactivating cancer cells is irradiation. For example, the cancer cells could be irradiated with between about 30 Gy and about 300 Gy using a cell irradiator for 30 minutes. Other methods of inactivating cancer cells include the use of cytotoxic agents or cytostatic agents. UV light could also be used to inactivate the cells. Yet another strategy for inactivating the transfected cancer cells would be to co-transfect the cancer cells with a suicide gene, such as HSV-TK. A cancer cell transfected with HSV-TK could then be killed after it was administered to the subject by giving the subject ganciclovir.

### E. THERAPEUTIC PROTEINS

The transfected cancer cells of the present invention are modified to express one or more therapeutic proteins. A “therapeutic protein” is a protein that can be administered to a subject for the purpose of treating or preventing a disease. For example, a therapeutic protein can be a protein administered to a subject for treatment or prevention of cancer. A therapeutic protein can be directly administered to a cell or subject, or it can be expressed from a nucleic acid molecule that is administered to the cell or subject. Examples of classes of therapeutic proteins include tumor suppressors, inducers of apoptosis, cell cycle regulators, immuno-stimulatory proteins, toxins, cytokines, enzymes, antibodies, inhibitors of angiogenesis, metalloproteinase inhibitors, hormones or peptide hormones.

1. Immuno-Stimulatory Proteins

In some embodiments of the invention, the therapeutic protein is an immuno-stimulatory protein. An “immuno-stimulatory protein” is a protein involved in the activation, differentiation, or chemotaxis of immune cells. Examples of classes of immuno-stimulatory proteins include cytokines and thymic hormones. Cytokines are described in more detail below. Thymic hormones include, for example, prothymosin-α, thymulin, thymic humoral factor (THF), THF-γ-2, thymocyte growth peptide (TGP), thymopoietin (TPO), thymopentin, and thymosin-α-1.

2. Cytokines

In one embodiment, the present invention provides methods and compositions for eliciting an enhanced immune cell-mediated killing of cancer cells. More specifically, the enhanced immune response is achieved by transfecting cancer cells with a nucleic acid molecule encoding a cytokine and administering the transfected cancer cell to a subject.

The term cytokine refers to a diverse group of secreted, soluble proteins and peptides that mediate communication among cells and modulate the functional activities of individual cells and tissues. Classes of cytokines include interleukins, interferons, colony stimulating factors, and chemokines. Examples of cytokines include: IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, leukocyte inhibitory factor (LIF), IFN-α, IFN-γ, TNF, TNF-α, TGF-β, G-CSF, M-CSF, and GM-CSF.

Interleukins are involved in processes of cell activation, cell differentiation, proliferation, and cell-to-cell interactions. Those of skill in the art are familiar with interleukins including, but not limited to: IL-1, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-17B, IL-17C, IL-17E, IL-17F, IL-18, IL-19, IL-20, IL-21, IL-21, IL-22, IL-23, IL-24, IL-25, IL-26, IL-27, IL-28A, IL-28B, IL-29, and IL-30.

Interferons are proteins that possess antiviral, antiproliferative, and immunomodulating activities. In addition, interferons influence metabolism, growth, and differentiation of cells. IFN-α, IFN-β, and IFN-γ are the three main human interferons. IFN-γ, which is produced primarily by the Th1 type of lymphocytes, exhibits many immunoregulatory effects, including the ability to induce the differentiation and activation of T cells and macrophages.

Colony stimulating factors include, for example, G-CSF, M-CSF, GM-CSF, IL-3, and MEG-CSA. Of these, GM-CSF has probably been used the most in studies on eliciting an enhanced immune cell-mediated killing of cancer cells.

Chemokines are a family of pro-inflammatory activation-inducible cytokines, which are mainly chemotactic for different cell types. There are four major classes of chemokines: C-chemokines, CC-chemokines, CXC-chemokines, and CX3C-chemokines. Non-limiting examples of chemokines include MCP-1, MCP-2, MCP-3, MIP-1α/β, IP-10, MIG, IL-8, RANTES, and lymphotactin.

Several cytokines, such as IL-2, IL-12, IL-15, IL-18, IL-21, GM-CSF, and IFNγ, have been studied for their ability to enhance immune cell-mediated killing of cancer cells. IL-2 has been shown to reduce tumorigenicity and metastatic potential of B16 melanoma (Karp et al, 1993), CMS-5 fibrosarcoma (Gansbacher et al., 1990), and murine bladder tumor (MBT-2) carcinoma (Connor et al., 1993). Human IL-2 and murine IL-12 co-transfected into KB cells using herpes virus and retrovirus vectors, respectively, has been shown to inhibit the growth of established tumors in a nude mouse model of head and neck squamous cell carcinoma (Kimura et al., 2003).

IL-21 is a cytokine produced mainly by activated T cells with effects that include costimulation of T cell proliferation, potentiation of NK cell maturation from bone marrow progenitors, and activation of peripheral NK cells (Kasaian et al., 2002). It has been proposed that IL-21 is a key element in the transition between innate and adaptive immune responses (Kasaian et al., 2002). IL-21 is related to IL-2, IL-4, and IL-15. Its cellular effects are mediated through IL-21R, a class I cytokine family receptor. Kishida et al. (2003) reported a synergistic, anti-tumor effect when IL-21 and IL-15 expression plasmid were intravenously injected in the tail veins of mice.

IL-15 is a cytokine produced primarily by macrophages. It is important for peripheral T cell maturation (Strengell et al. 2003). IL-18 is another primarily macrophage-derived cytokine. It is an important cofactor in IFN-γ gene activation. Macrophage-derived IFN-α and IL-12 have also been shown to be important regulators of IFN-γ gene expression. It has been shown that IL-15, IL-18, and IL-21 act synergistically in activating early NK cell responses (Strengell et al., 2003). Kishida et al. (2001) have reported that in vivo electroporation-mediated transfer of IL-12 and IL-18 genes induced anti-tumor effects against melanoma in mice (Kishida et al., 2001).

GM-CSF is a growth factor for monocytes and neutrophils, activates macrophages, and promotes differentiation of dendritic cells. Dranoff et al. was one of the first to use B16 melanoma cells transduced with GM-CSF to treat melanoma (Dranoff et al., 1993).

In one embodiment the present invention provides a method of treating cancer in a subject comprising: obtaining a cancer cell composition; transfecting cancer cells of the composition by electroporation with one or more nucleic acid molecules encoding one or more cytokines; and administering the transfected cells to the subject. It is contemplated that the cancer cell may be transfected with a single cytokine or with a combination of cytokines. A combination of cytokines that act on multiple cell types may result in a more robust immune response to the cancer cell.

For example, a tumor cell modified to express IL-21, IL-15, and GM-CSF could be used. As described above, IL-15 in synergy with IL-21 promotes NK and T cell activity. Also as described above, GM-CSF is a growth factor for monocytes and neutrophils, activates macrophages, and promotes differentiation of dendritic cells. Transfected cancer cells expressing GM-CSF will attract APCs, such as dendritic cells, which will phagocytize the transfected cancer cells and process/present them to the nearby T cells. Thus, by combining cytokines that act on multiple cell types, the present invention provides methods and composition that enable a more effective immune response to cancer cells. Moreover, with this approach it does not matter if the tumor antigens are unknown, since the transfected cancer cells would be expected to present a wide range of tumor cell antigens to the immune cells.

3. Other Immuno-Stimulatory Proteins

Other immuno-stimulatory proteins that may be used in the methods and compositions of the present invention include B7.1 (CD80), B7.2 (CD86), CD40, CD40 Ligand (CD40L), LFA-1, ICAM-1, VLA-4, and VCAM-1. CD40L is a co-stimulator molecule for multiple components of the immune response. It is an approximately 35 kDa glycoprotein of 261 amino acids and a member of the tumor necrosis factor superfamily. CD40L is expressed on activated T cells, mostly CD4+ but also some CD8+ and basophils/mast cells. CD40L binds to CD40, an integral membrane protein found on the surface of B lymphocytes, dendritic cells, follicular dendritic cells, hematopoietic progenitor cells, epithelial cells, and carcinomas.

CD40-CD40L interactions play a key role in B-cell activation and differentiation, augmentation of the antigen presenting function of B cells and professional antigen presenting cells (APC), and stimulation of CD4+ and CD8+ T cells that have become activated by engagement of antigen on APCs.

While leukemia cells may express tumor-specific antigens in association with Class I and II MHC molecules, they often lack expression of conventional co-stimulator molecules necessary to induce T cell activation. Dilloo et al. (1997) demonstrated that injection of otherwise non-immunogenic A20 (CD40+ murine lymphoblastic leukemia) cells in the presence of CD40L induced an immune response active against a pre-existing A20 tumor at a distant cite. In addition, concomitant local secretion of transgenic IL-2 further amplified the anti-leukemic response (Dilloo et al. (1997)).

Forced expression of CD40L via adenovirus based vector was reported to upregulate the important co-stimulatory molecules on the B-CLL cell surface and transform the B leukemia cells to antigen presenting cells and induce an autologous immune recognition of the B-CLL cells in vitro and in patients (Wierda et al. 2000). However, transduction by recombinant adenovirus requires an extremely high MOI (multiplicity of infection, virus particles per cell) because B-CLL cells and other cancer cells lack the essential Coxsackie and Adenovirus Receptor (CAR). Previous phase I clinical study results showed that 2000 adenovirus particles were needed to transduce 1 B-CLL cell (Wierda et al. 2000).

B7.1 is a membrane glycoprotein of 262 amino acids. B7.1 is expressed primarily on activated B-cells and other antigen-presenting cells. It is expressed by macrophages, keratinocytes, T-cells, B-cells, peripheral blood dendritic and Langerhans cells. B7.2 is found on blood dendritic and Langerhans cells, B-cells, macrophages, Kupffer cells, activated monocytes and various natural killer cell clones.

B7.1 is a ligand of CD28. Binding of B7 to CD28 on T-cells delivers a co-stimulatory signal that triggers T-cell proliferation by stimulating a transcription factor that, in turn, induces the synthesis and secretion of IL-2 and other cytokines.

B7.1 binds to another protein structurally related to CD28, called CTLA-4 (cytotoxic T-lymphocyte associated antigen 4). CTLA-4 is expressed in low-copy number by T-cells only after activation, but it binds B7.1 with approximately 20-fold higher affinity than CD28.

Adhesion molecules, such as ICAM-1, VCAM-1, LFA-1, and VLA-4, could also be used in the context of the present invention. ICAM-1 and VCAM-1 are immunoglobulins. LFA-1 and VLA-4 are integrins. Interaction of LFA-1 (lymphocyte function-associated antigen-1) with ICAM-1 (intercellular adhesive molecule-1) is important in a number of cellular events, including inflammation, adhesion, and transendothelial migration. The interaction of VCAM-1 (vascular cell adhesion molecule 1) with VLA-4 (very late activation antigen 4) is important for lymphocyte/endothelial interactions at inflammatory sites.

### F. NUCLEIC ACID-BADED EXPRESSION SYSTEMS

1. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Goodburn and Maniatis et al., 1988 and Ausubel et al., 1996, both incorporated herein by reference).

The term “expression vector” refers to any type of genetic construct comprising a nucleic acid coding for an RNA capable of being transcribed and then translated into a protein, polypeptide, or peptide. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles, such as mitochondria, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 2001, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 1 lists non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart et |
| | | al., 1985; Imagawa et al., |
| | | 1987, Karin et al., 1987; |
| | | Angel et al., 1987b; |
| | | McNeall et al., 1989 |
| MMTV (mouse | Glucocorticoids | Huang et al., 1981; Lee et |
| mammary | | al., 1981; Majors et al., |
| tumor virus) | | 1983; Chandler et al., 1983; |
| | | Lee et al., 1984; Ponta et |
| | | al., 1985; Sakai et al., 1988 |
| β-Interferon | Poly(rI)x | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor α | | |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Non-limiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

The use of internal ribosome entry sites (IRES) elements may be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to be more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a cell. In general, plasmid vectors containing replicon and control sequences which are derived from species compatible with the cell are used in connection with these cells. The vector ordinarily carries a replication site, as well as marking sequences which are capable of providing phenotypic selection in transformed cells.

### H. CELL CULTURE

In certain embodiments of the invention, cell culture may be utilized in preparation of the cancer cells. In eukaryotic cell culture systems, the culture of the cells is generally under conditions of controlled pH, temperature, humidity, osmolarity, ion concentrations, and exchange of gases. Regarding the latter, oxygen and carbon dioxide are of particular importance to the culturing of cells. In a typical eukaryotic cell culture system, an incubator is provided in which carbon dioxide is infused to maintain an atmosphere of about 5% carbon dioxide within the incubator. The carbon dioxide interacts with the tissue culture medium, particularly its buffering system, in maintaining the pH near physiologic levels.

In addition to carbon dioxide, the culturing of cells is dependent upon the ability to supply to the cells a sufficient amount of oxygen necessary for cell respiration and metabolic function. Methods to increase oxygen concentration to the cultured cells include mechanical stirring, medium perfusion or aeration, increasing the partial pressure of oxygen, and/or increasing the atmospheric pressure.

Conventional cell culture containers comprise tissue culture flasks, tissue culture bottles, and tissue culture plates. Gas exchange between the incubator atmosphere and a tissue culture plate generally involves a loosely fitting cover which overhangs the plate. Similarly, for a tissue culture flasks or bottle, a loosely fitting cap excludes particulate contaminants from entering the chamber of the flask or bottle, but allows gas exchange between the incubator atmosphere and the atmosphere within the flask or bottle. Caps with a gas permeable membrane or filter are also available, thereby allowing for gas exchange with a tightly fitting cap.

### I. ACTIVATION AND EXPANSION OF B CELSS IN CULTURE

The present invention also provides methods and compositions for the activation and expansion of B cells in culture. The ex vivo activation and expansion of B cells is useful for a variety of applications including cellular therapy, immunotherapy, drug screening, and antigen screening. Although it is known that CD40L is capable of activating B cells, which allows B cell proliferation/expansion ex vivo, direct gene delivery of CD40L to B cells via viral vectors is extremely difficult.

Many groups tried using adenoviral vectors to deliver CD40L to B-CLL cells. However, due to the lack of the Coxsackie and Adenovirus Receptor (CAR) on the B-CLL cell surface, an enormously high MOI, e.g. >2000 virus particles/cell, was used in a human CLL clinical trial to transduce B-CLL cells with an adenoviral vector carrying mouse CD40L (Wierda et al. 2000). It was shown later that using a feeder cell line, e.g. MRC-5, could improve B-CLL transduction capability by adenoviral vector slightly. To further improve hCD40L gene delivery via adenovirus transduction, recently, Biagi et al., (2003) demonstrated that hCD40L could translocate from the MRC-5 feeder cell line to co-cultured B-CLL cells, when MRC-5 cells were genetically modified to express hCD40L constitutively.

Most recently, Wendtner et al. (2004) reported that adenovirus helper free rAAV could mediate efficient mCD40L expression in primary B-CLL cells at much lower MOI, however, mCD40L expressing HeLa cells were again used as feeder cells during rAAV transduction procedure. The requirement of CD40L expressing feeder cells modified via an adenovirus or rAAV transduction protocol is not optimal for CLL or any other cancer immunotherapy. Though the feeder cells were γ-irradiated before co-culturing with primary B-CLL cells, ideally, they should be removed from the cancer vaccine product prior to administration to patients. Providing evidence that the autologous cancer vaccine is feeder cell free requires extensive testing, moreover, it is known that a certain percentage of the product will be lost during removal of the contaminated feeder cells. Furthermore, establishing and maintaining a master bank of CD40L expressing feeder cells is labor and time consuming.

An NIH 3T3 derived cell line that constitutively expresses hCD40L on the cell surface has been used for human B cell expansion for preclinical studies; however, this mouse cell line has not been approved for human clinical studies. A recombinant hCD40L trimer has been reported to be a potential molecule for ex vivo expansion of B cells; however, the use of a recombinant hCD40L raises issues such as the purity of the protein, complications of protein production, and protein stability.

The present invention overcomes these difficulties through electroporation-mediated direct transfection of CD40L DNA, which provides efficient and fast expression of CD40L in primary B cells. Expression of CD40L upregulates the expression of immuno co-stimulatory molecules, e.g. CD80 and CD86. Furthermore, when B cells that are genetically modified to express CD40L are mixed with unmodified B cells, the co-stimulatory molecules were also upregulated on the unmodified B cells. Thus, B cells or PBMC (peripheral blood mononuclear cells) expressing CD40L can be used as a source of CD40L for unmodified B cells, which will allow naïve B cells to be activated and expanded ex vivo.

In certain aspects of the invention, the PBMC or purified B cells are obtained from a subject and then divided in to two parts, one for expansion and one for transfection with CD40L. The cells transfected with CD40L can then be used to activate the untransfected cells. The cells transfected with CD40L may be frozen, and may then be thawed and mixed with the untransfected cells. In this example, the transfected cells and the untransfected cells are autologous, thus reducing complications in applications where the cells are reintroduced into the subject. In one embodiment of the invention, CD40L-transfected B-CLL cells, either alone or mixed with untransfected B-CLL cells may be used as a vaccine in patients with leukemia.

### J IMMUNODETECTION METHODS

In certain embodiments, the present invention concerns immunodetection methods for measurement of the immune response against the transfected cancer cells. Immunodetection methods can also be used to verify transgene expression in genetically modified cancer cells. One of ordinary skill in the art would be familiar with a wide variety of immunodetection techniques that are available. Examples of immunodetection methods include enzyme linked immunosorbent assay (ELISA), ELISpot, radioimmunoassay (RIA), immunoradiometric assay, fluoroimmunoassay, chemiluminescent assay, bioluminescent assay, and Western blot, to mention a few. The steps of various useful immunodetection methods have been described in the scientific literature, such as, e.g., Doolittle and Ben-Zeev, 1999; Gulbis and Galand, 1993; De Jager et al., 1993; and Nakamura et al., 1987, each incorporated herein by reference.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA, incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

### K. PHARMACEUTICAL PREPARATIONS

1. Formulations

Pharmaceutical preparations of cancer cells modified to express therapeutic proteins for administration to a subject are contemplated by the present invention. One of ordinary skill in the art would be familiar with techniques for administering cells to a subject. Furthermore, one of ordinary skill in the art would be familiar with techniques and pharmaceutical reagents necessary for preparation of these cell prior to administration to a subject.

In certain embodiments of the present invention, the pharmaceutical preparation will be an aqueous composition that includes the transfected cancer cells that have been modified to express one or more therapeutic proteins. In certain embodiments, the transfected cancer cell is prepared using cancer cells that have been obtained from the subject. However, cancer cells obtained from any source are contemplated by the present invention. The cancer cells may have been obtained as a result of previous cancer surgery performed on the subject as part of the overall cancer treatment protocol that is specific for the particular patient.

It is desirable to inactivate the transfected cancer cells for use in pharmaceutical preparations. The transfected cancer cells can be inactivated prior to administering them to the subject by, for example, irradiating the cells, or contacting the cells with a cytostatic agent or a cytotoxic agent.

Aqueous compositions of the present invention comprise an effective amount of a solution of the transfected cancer cells in a pharmaceutically acceptable carrier or aqueous medium. As used herein, “pharmaceutical preparation” or “pharmaceutical composition” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the transfected cancer cells, its use in the therapeutic compositions is contemplated. Supplementary active ingredients can also be incorporated into the compositions. For human administration, preparations should meet sterility, pyrogenicity, general safety and purity standards as required by the FDA Center for Biologics.

The biological material should be extensively dialyzed to remove undesired small molecular weight molecules and/or lyophilized for more ready formulation into a desired vehicle, where appropriate. The transfected cancer cells will then generally be formulated for administration by any known route, such as parenteral administration. Determination of the number of cells to be administered will be made by one of skill in the art, and will in part be dependent on the extent and severity of cancer, and whether the transfected cancer cells are being administered for treatment of existing cancer or prevention of cancer. The preparation of the pharmaceutical composition containing the transfected cancer cells of the invention disclosed herein will be known to those of skill in the art in light of the present disclosure.

The present invention contemplates cancer cells transfected to express one or more therapeutic proteins that will be in pharmaceutical preparations that are sterile solutions for subcutaneous injection, intramuscular injection, intravascular injection, intratumoral injection, or application by any other route. A person of ordinary skill in the art would be familiar with techniques for generating sterile solutions for injection or application by any other route.

Upon formulation, solutions will be administered in a manner compatible with the dosage formulation and in such amount as is therapeutically effective. The formulations are easily administered in a variety of dosage forms, such as the type of injectable solutions described above. For parenteral administration, the solution including the transfected cancer cells should be suitably buffered. The transfected cancer cells may be administered with other agents that are part of the therapeutic regiment of the subject, such as other immunotherapy or chemotherapy.

2. Dosage

The present invention contemplates administration of cancer cells transfected to express one or more therapeutic proteins to a subject for the treatment and prevention of cancer. An effective amount of the transfected cancer cells is determined based on the intended goal, for example tumor regression. For example, where existing cancer is being treated, the number of cells to be administered may be greater than where administration of transfected cancer cells is for prevention of cancer. One of ordinary skill in the art would be able to determine the number of cells to be administered and the frequency of administration in view of this disclosure. The quantity to be administered, both according to number of treatments and dose, also depends on the subject to be treated, the state of the subject, and the protection desired. Precise amounts of the therapeutic composition also depend on the judgment of the practitioner and are peculiar to each individual. Frequency of administration could range from 1-2 days, to 2-6 hours, to 6-10 hours, to 1-2 weeks or longer depending on the judgment of the practitioner.

Longer intervals between administration and lower numbers of cells may be employed where the goal is prevention. For instance, numbers of cells administered per dose may be 50% of the dose administered in treatment of active disease, and administration may be at weekly intervals. One of ordinary skill in the art, in light of this disclosure, would be able to determine an effective number of cells and frequency of administration. This determination would, in part, be dependent on the particular clinical circumstances that are present (e.g., type of cancer, severity of cancer).

In certain embodiments, it may be desirable to provide a continuous supply of the therapeutic compositions to the patient. Continuous perfusion of the region of interest (such as the tumor) may be preferred. The time period for perfusion would be selected by the clinician for the particular patient and situation, but times could range from about 1-2 hours, to 2-6 hours, to about 6-10 hours, to about 10-24 hours, to about 1-2 days, to about 1-2 weeks or longer. Generally, the dose of the therapeutic composition via continuous perfusion will be equivalent to that given by single or multiple injections, adjusted for the period of time over which the doses are administered.

### L. COMBINATION TREATMENTS

In order to increase the effectiveness of the transfected cancer cells as a cancer therapy, it may be desirable to combine treatment using these cells with other agents or methods effective in the treatment of cancer. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the transfected cancer cells and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with immunotherapy. In the context of the present invention, it is contemplated that the transfected cancer cells could be used similarly in conjunction with chemotherapeutic, radiotherapeutic, or other immunotherapeutic intervention.

Alternatively, the immunotherapy with transfected cancer cells may precede or follow the other treatment by intervals ranging from minutes to weeks. In embodiments where the other agent and the transfected cancer cells are applied separately to the subject, one would generally ensure that a significant period of time did not expire between the time of each delivery, such that the agent and transfected cancer cells would still be able to exert an advantageously combined effect on the subject. In such instances, it is contemplated that one may contact the subject with both modalities within about 12-24 h of each other and, more preferably, within about 6-12 h of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Various combinations may be employed, immunotherapy is “A” and the secondary agent, such as radio- or chemotherapy, is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

It is expected that the treatment cycles would be repeated as necessary. It also is contemplated that various standard therapies, as well as surgical intervention, may be applied in combination with the described therapy using transfected cancer cells.

1. Chemotherapy

Cancer therapies include a variety of combination therapies with both chemical and radiation based treatments. One of ordinary skill in the art would be familiar with the range of chemotherapeutic agents and combinations that are available. Chemotherapeutic agents include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

2. Radiotherapy

Other factors that cause DNA damage and have been used extensively include γ-rays, X-rays, and the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

3. Immunotherapy

The transfected cancer cells of the present invention may be administered in combination with other forms of immunotherapy. Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Antigen presenting cells (APCs), such as dendritic cells, loaded with cancer cell lysate may also be used in combination with the transfected cancer cells of the present invention.

4. Genes

The secondary treatment may be a gene therapy. For example, the gene therapy can be a vector encoding a tumor suppressor such as p53 or Rb.

5. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to the physical removal of at least part of a tumor. As previously noted, resected tumor cells can be used in the generation of the transfected cancer cells used in the treatment of the cancer patient. In addition to tumor resection, treatment by surgery includes laser surgery, cryosurgery, electrosurgery, and micrographic surgery (Mohs' surgery).

Upon excision of part or all of the cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, or 5 weeks or every 1, 2, 3, 4, 5, 6, 7, 8, 9, 10, 11, or 12 months. These treatments may be of varying dosages as well.

6. Other Agents

It is contemplated that other agents may be used in combination with the present invention to improve the therapeutic efficacy of treatment. These additional agents include other immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers.

It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases in intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion, such as integrin and cadherin blocking antibodies, are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### M. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Transfection of renal carcinoma cells (RENCA) was optimized using the marker gene eGFP containing DNA plasmid. Under optimal conditions, greater than 80% of the transfected RENCA cells expressed eGFP as analyzed by FACS 24 hours post electroporation.

Mouse IL-12, IL-21, IL-15 and GM-CSF full-length cDNA were each subcloned into a commercially available DNA plasmid, pVAX (Invitrogen), in which the transgene is regulated by the CMV promoter.

Young Balb/C mice (˜8 week of age) from Jackson Laboratory were first injected subcutaneously with 5e5 RENCA cells, on their left side. Seven days later, mice with established tumors were randomly divided into 5 groups, 8 mice for each group. Then the mice were subcutaneously injected on their right side (remote from primary tumor site) one time with 5e5 electroporated RENCA cells, which were transfected with the cytokine combinations as following: IL-12/IL-21/GM-CSF, IL-12/IL-15/IL-21, IL-12/IL-15/GF-CSF, and IL-15/IL-21/GM-CSF; the control group received tumor cells electroporated but without DNA.

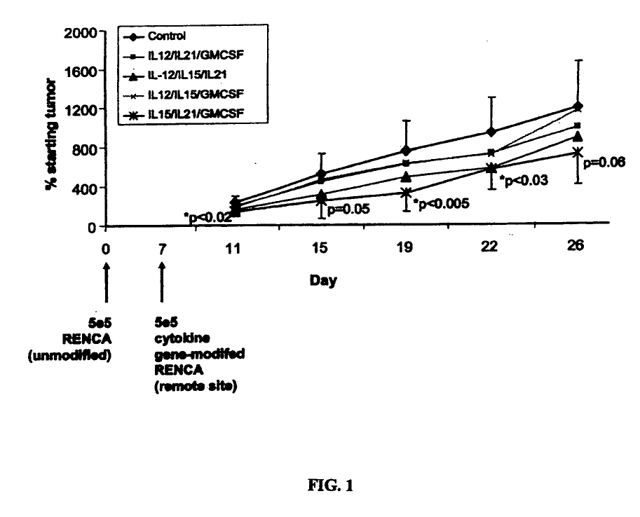

The primary tumor size was measured and normalized by its original size on day 7. As shown in FIG. 1, all of the RENCA cells electroporated with various combinations of cytokines demonstrated slower primary tumor growth relative to the control. The IL-15/IL-21/GM-CSF modified RENCA cells showed the most significant inhibition of primary tumor growth at all time points.

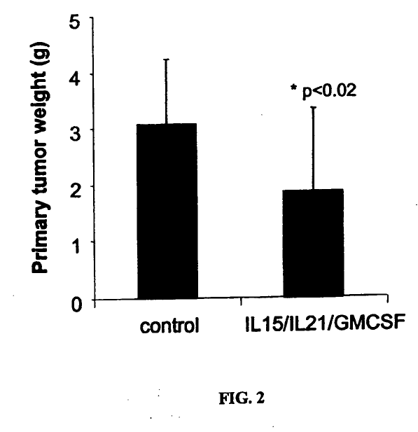

Mice were sacrificed at day 26 following IACUC guidance. Primary tumors were removed from the control group and the IL-15/IL-21/GM-CSF group. Weighing of the primary tumors revealed significantly smaller tumors from IL-15/IL-21/GM-CSF modified RENCA treatment (FIG. 2, p<0.02).

### Example 2

Further experiments were performed to evaluate the anti-tumor effect of RENCA cells modified with GM-CSF, IL-15 and IL-21, alone and in various combinations. As in Example 1, primary tumors were established in Balb/C male mice by sub-cutaneous injection with 5e5 unmodified RENCA cells. Each mouse was ear-tagged to allow for continuous monitoring. Injections were administered in the rear, left backs of shaved mice. The tumors typically follow a predictable progression, being detectable by day 5 and readily measured by day 6-7. On day 7, tumors were measured by digital calipers and only those mice with statistically identical tumor areas were used. This ensures an equivalent baseline tumor area. Mice were then sorted into 9 groups of 10 mice.

RENCA cells were electroporated with various combinations of plasmid DNA encoding cytokine genes. The genes selected for this study were mGM-CSF, mIL-15 and mIL-21. Each transgene was contained on identical plasmid backbones containing EBNA1 and oriP elements for enhanced transient gene expression. A total of 9 transfection groups were included:

(1) Electroporation with no DNA control

(2) GM-CSF/IL-15/IL-21

(3) IL-15/IL-21

(4) GM-CSF/IL-15

(5) GM-CSF/IL-21

(6) GM-CSF only

(7) IL-15 only

(8) IL-21 only

(9) Electroporation with empty vector control

The transfected RENCA cells were plated overnight in T175 tissue culture flasks to allow for gene expression. Transgene expression was confirmed in vitro by ELISA analysis of the cell culture supernatants. After 24 hours, the RENCA cells were collected by trypsinization, washed with PBS, and counted.

Coinciding with day 7 post primary tumor cell injection, the same Balb/C mice were injected on the opposite backside (rear, right) with 5e5 gene-modified RENCA cells. A total of 90 mice were used: 10 mice/group.

The area of the established tumors were measured twice per week for a total of 3 weeks using digital calipers. Results were adjusted for each mouse as follows:

$$\frac{Tumor area at day XX}{Starting tumor area at day 7} \times 100 \%$$

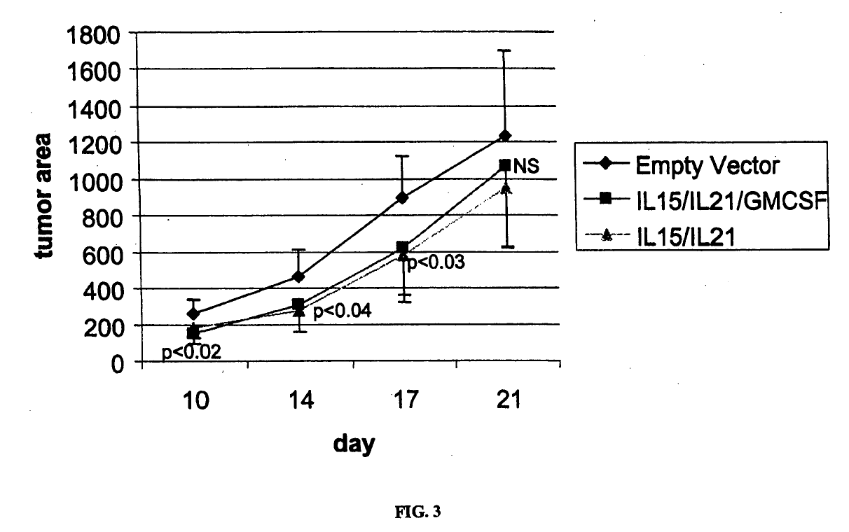

FIG. 3 shows a graph of the tumor areas for mouse group 9 (empty vector control), group 2 (GM-CSF/IL-15/IL-21), and group 3 (IL-15/IL-21). The co-expression of IL-15 and IL-21, either with or without GM-CSF, elicited a significant reduction in tumor area. The addition of GM-CSF did not significantly effect the tumor area (i.e., group 2 and 3 are not statistically different from each other). The no DNA control (group 1) was indistinguishable from the empty vector control (group 9).

No cytokine by itself (groups 6, 7, 8) had a significant effect on tumor area relative to controls. Thus, IL-15 and IL-21 appear to be synergizing for an anti-tumor effect.

### Example 3

Materials and Methods:

B-CLL Cells. After informed consent was obtained, peripheral blood was collected from CLL patients at Washington Cancer Institute (Washington, D.C.) or at Baylor College of Medicine (Houston, Tex.). B-CLL cells were isolated by standard Ficoll Paque gradient separation procedure. Briefly, the total peripheral blood was first diluted with equal volume of PBS (BioWhittaker, Md.) containing 10 mM NaCitric (Sigma, St. Louis, Mo.) prior to being layered atop of Ficoll Paque in a 50 mL conical tube. After 20 minutes centrifugation at 160×g without braking, the cells at the interphase were collected, washed twice with PBS, and then cryopreserved. A small fraction of the cells were saved and characterized by flow cytometry. Generally, FACS analysis of cell surface markers revealed greater than 90% of the cell population were CD5/CD19 double positive.

Antibodies and Reagents. Fluorescein isothiocyanate (FITC)-conjugated monoclonal antibody (Mab) specific for IgG1, hCD40L and MHC II (HLA-DR); phycoerythrin (PE)-conjugated Mab specific to hCD54 (ICAM-1), hCD80 (B7-1) and hCD86 (B7-2); Cychrom-conjugated Mab specific to hCD5 were purchased from BD Pharmogen. FITC labeled dextran (500 kD) was obtained from Sigma (St. Louis, Mo.).

Plasmids. Full-length cDNA encoding for hCD40L and hIL2 was amplified from a human leukocyte cDNA library (Clontech, San Josa, Calif.) by PCR using primers engineered with an NheI restriction enzyme digestion site at the 5′ end, and a NotI digestion site at the 3′ end. After enzyme digestion and gel cleaning, the PCR fragment was subcloned into the same restriction sites on the pDsRed-N1 (Clontech, San Josa, Calif.) backbone to replace the DsRed transgene, which is regulated by the CMV promoter. Both subcloned hCD40L and hIL2 were sequenced to confirm nucleotide sequence with the one in public domain (PubMed). The phCD40L and phIL-2 plasmids were manufactured by Althea Technologies (San Diego, Calif.) following current Good Manufactory Practice (cGMP) guideline that the entire construct was sequenced 3 times to exclude any mutated nucleotides.

The peGFP plasmid was constructed as previously described (Li et al. 2002) and was propagated in E coli strain DH5α (Invitrogen) and purified on endotoxin-free Qiagen-tip 10000 columns (Qiagen, Chatsworth, Calif.). Each batch of plasmid DNA was routinely checked for its A260/A280 ratio (1.75 to 1.9), endotoxin level (3 to 22 EU/mg), and percentage of super coil population (80 to 95%).

Electroporation Based Non-Viral Gene Delivery. Cryopreserved B-CLL cells were thawed following standard procedure and later were incubated in 37° C. pre-warmed complete culture medium (10% FBS in RPMI-1640, 2mM L-glutamine) for 30 minutes (unless specified otherwise). The CLL-B cells were then washed one time with electroporation (EP) buffer (Hyclone). The washed CLL-B cells were resuspended in EP buffer at a cell concentration from Xe7 to Xe8 cells/mL, together with plasmid DNA at a final concentration of 440 μg/mL or 0.5 mg/mL of FITC-dextran. The cell mixture was then transferred to either a MaxCyte standard microcuvette by a micropipettor or a clinical grade (CL-1) processing chamber by a syringe. After docking the chamber onto a MaxCyte GT electroporator, the cells were electroporated with various pulses at a variety of field strengths (1 kV to 3 kV/cm) and a range of pulse width (10 μs to 10 ms). The processed CLL-B cells were then transferred to a clean tube. After incubation at 37° C. for 20 minutes, the transfected CLL-B cells were cultured in complete media. Transgene expression and cell viability were examined at various time points post electroporation by FACS analysis.

Flow Cytometry. Transgene hCD40L expression and other cell surface markers were analyzed by flow cytometry analysis. The cells at various time points post transfection were harvested by centrifugation and washed with PBS one time. The cells were then incubated with specific, fluorescence-conjugated Mab, and propidium iodine (PI) for 20 minutes at 4° C. followed by one PBS wash. The labeled cells were examined by FACSCalibur™ (BD Biosciences) with proper gating using isotype Mab labeled cells as control. Gating was set at ≦0.5% of the control cells to be fluorescent positive cells. Viability was calculated by PI exclusion. Trypan blue exclusion by light microscope has also been used for viability analysis.

Cryopreservation. Cells were cryopreserved in 10% DMSO in FBS.

Transactivation Assay. Primary B-CLL cells were first electroporated with FITC-Dextran (500 kD) following standard transient transfection procedure. After 3 times PBS washing, FITC-Dextran containing, control B-CLL cells were co-cultured with an equal amount of hCD40L-transfected B-CLL cells from the same donor at 37° C. for 24 hours. Then the total mixture was incubated with PE-conjugated anti-CD86 or PE-conjugated anti-hCD40L Mab followed by FACS analysis for expression of CD86 and hCD40L indicated by FITC and PE double positive cells.

Mixed Lymphocyte Reaction. HLA non-matched, allogeneic lymphocytes, were obtained from leukapheresis product by Ficoll Paque gradient isolation and later purified by removal of the attached cells in T175 flask. In a well on a 96 well plate, 4e5 of the allogeneic lymphocytes were mixed with 2e5 hIL2-transfected B-CLL cells and 4e5 hCD40L-transfected B-CLL cells, or control B-CLL cells. After co-culturing for 40 to 48 hours, the conditioned culture media was removed and analyzed by a commercially available ELISA kit (R&D System) for IFN-γ production. The standard deviation was given from 4 repeated experiments with a p value at p<0.001.

Statistical Analysis. Unpaired student-t test with two tails was used to determine the significance of results. Data were presented as mean i standard deviation.

Results:

Efficient gene delivery of marker gene and hCD40L to primary B-CLL cells. A standard DNA plasmid carrying a full-length cDNA encoding for the enhanced green fluorescence protein (eGFP) marker gene was used to optimize the transient transfection procedure for B-CLL cells. Numerous experiments were performed to test various cell handling procedures, electroporation parameters, DNA and cell concentration and other factors. eGFP-transfected B-CLL cells showed strong eGFP expression while maintaining good cell morphology. Transgene expression was rapid, being observed within a few hours post transfection. When the eGFP transfected B-CLL cells were analyzed by flow cytometry, 52% of the processed cells expressed the eGFP marker gene.

Transient transfection of B-CLL cells was also examined with the DNA plasmids carrying the full-length cDNA encoding for human CD40L instead of eGFP. Electroporation was able to mediate efficient and rapid hCD40L expression. When the transfected B-CLL cells were analyzed by FACS at 3 hours post transfection approximately 56% of CD5/CD19 double positive B-CLL cells expressed hCD40L.

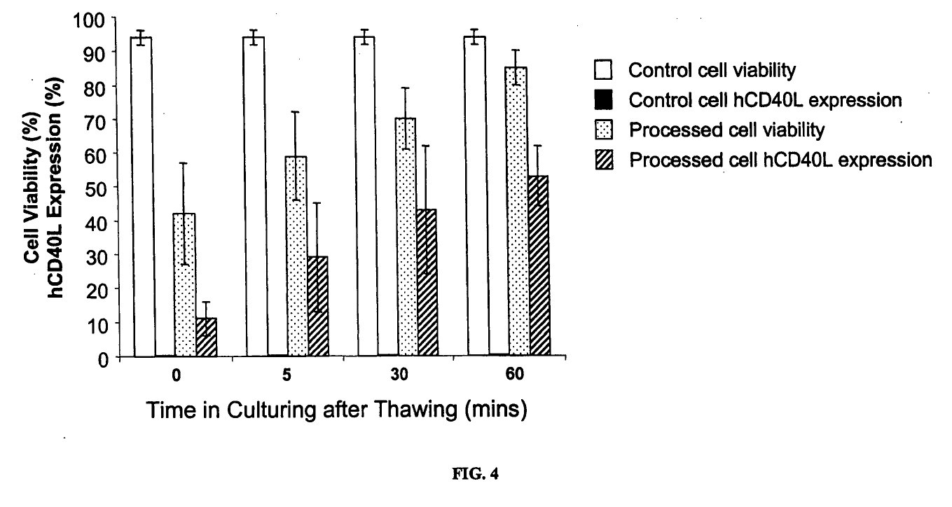

Optimization of transfection of cryopreserved B-CLL cells. Though ideally, freshly isolated B-CLL cells are optimal for processing, cryopreserved cells are routinely used in clinical settings. During optimization, it was investigated how soon the cryopreserved B-CLL cells could be transfected after thawing. Cryopreserved B-CLL cells were thawed and then cultured in 37° C. CO₂ incubators. At various time points, 0, 5, 30, and 60 minutes, cultured cells were harvested and electroporated with the phCD40L plasmid. As shown in FIG. 4, culturing the thawed cells prior to transfection increased both cell viability and hCD40L expression.

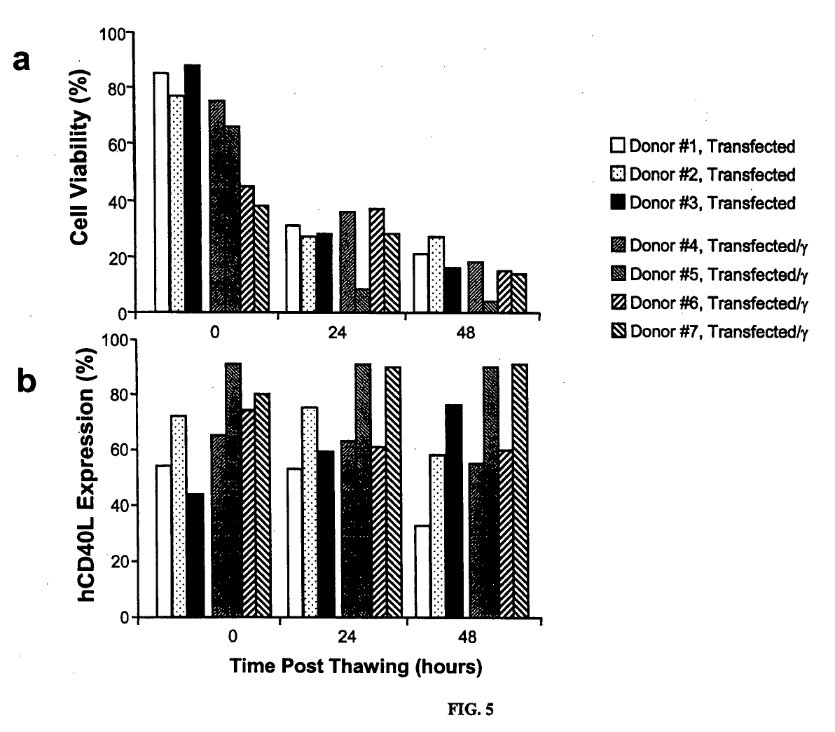

Consistent non-viral gene delivery to primary B-CLL cells. After optimization of the CLL-B cell process procedure, samples from 7 CLL patients (donors #1 to #7) were processed. All patients' cells were cryopreserved before transfection. Data presented in FIG. 5 shows good cell viability of hCD40L-transfected B-CLL cells immediately after thawing. Viability declined after 24 hours in culture, from 70% just after thawing to 25% at 24 hours.

The viability of cryopreserved cells just after thawing is similar to that of cells that were not cryopreserved, indicating that electroporation caused very low level of physical damage on the cells. The decreased viability of the long-term cultured cells (>24 hrs) but not the short-term cultured hCD40L-transfected cells might be due to apoptosis. The same phenotype was observed on eGFP-transfected B-CLL cells suggesting apoptosis was not induced by the transgene itself. Electroporation of the B-CLL cells with FITC-dextran did not cause apoptosis. The decreased viability of the long-term cultured cells also did not appear to be related to the γ-irradiation. To test whether the decreased viability of the long-term cultured cells was due to apoptoses, the hCD40L-transfected B-CLL cells were immunostained with FITC-conjugated VAD-FMK (FITC-VAD-FMK) at 48 hours post thawing. FITC-VAD-FMK positive cells were detected by FACS analysis indicating that these cells were apoptotic.

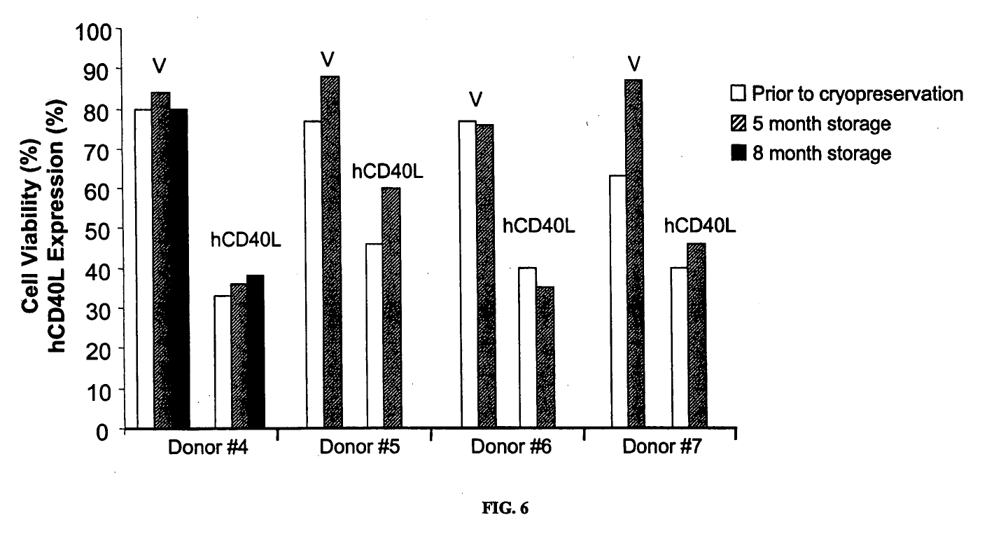

Cryopreserved hCD40L-transfected B-CLL cells are stable. The stability of hCD40L-transfected B-CLL cells after long-term storage in liquid nitrogen was also examined. FACS analysis showed no significant changes in cell viability and hCD40L expression of the cryopreserved, hCD40L-transfected cells after five months storage in liquid nitrogen (FIG. 6). The transfected cells from one donor (donor #4) were stored up to 8 months, and there was no alteration detected for cell viability and hCD40L expression.

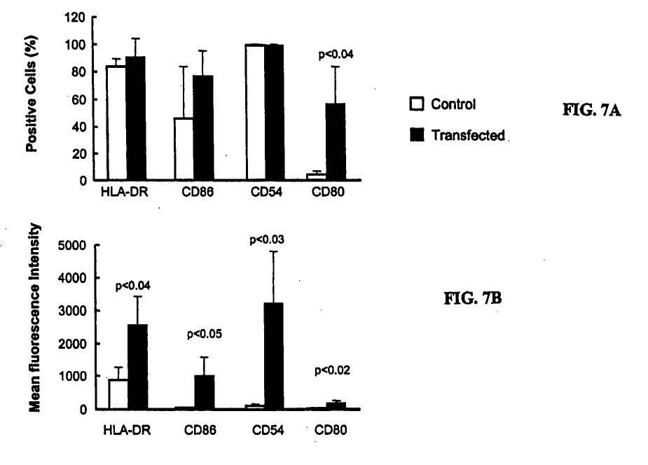

hCD40L upregulated immuno accessory gene expression in transfected B-CLL cells. Immuno accessory gene expression was analyzed on the hCD40L-transfected B-CLL cells at 48 hours post thawing by first immunostaining with FITC-conjugated Mab specific for hCD40L, HLA-DR, CD86, CD80, and CD54 followed by FACS analysis. FIGS. 7A and 7B summarize the quantified results of the up-regulation of HLA-DR, CD80, CD86 and CD54 molecules on hCD40L-transfected cells from three B-CLL donors. Although, the percentage of HLA-DR, CD86 and CD54 positive cell population did not increase significantly after hCD40L transfection (FIG. 7A), the expression level of HLA-DR, CD86 and CD54 increased dramatically as indicated by the mean fluorescence intensity of FITC-conjugated Mab (p value<0.04, 0.05, and 0.03 respectively, FIG. 7B). Furthermore, both the percentage of CD80+ cells and the expression level of CD80 were significantly higher in the hCD40L-transfected cells than in the control cells, 57%±27% vs. 4.3%±2.5% (p<0.04). Data presented here demonstrated that hCD40L extensively upregulated gene expression of the immuno accessory molecules in the transfected B-CLL cells.

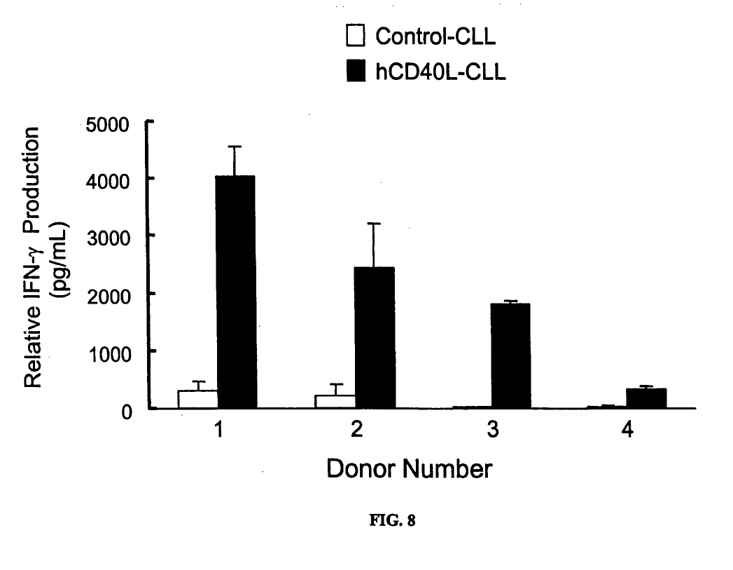

hCD40L-transfected B-CLL cells induced allogeneic immuno response. It is well known that B-CLL cells lack immunogenic capability in that they fail to trigger allogeneic T cell response. Forced expression of hCD40L in B-CLL cells can rescue their allogeneic function. To prove that the hCD40L-transfected B-CLL cells are functional, the transfected cells were mixed with allogeneic lymphocytes for 48 hours prior to analysis of the conditioned culture media for IFN-γ production. FIG. 8 illustrates IFN-γ production from allogeneic lymphocytes after co-culturing with cells from CLL patients, either mock transfected or transfected with hCD40L DNA plasmid together with hIL2-transfected B-CLL cells. A significant amount of (p<0.001) IFN-γ was observed from samples co-cultured with a combination of hCD40L and hIL2 transfected B-CLL cells. Moderate IFN-γ production was observed from samples co-cultured with hCD40L-transfected B-CLL cells alone, and minimum IFN-γ production was detected from samples co-cultured with hIL2-transfected B-CLL cells alone, suggesting that the rescue of allogeneic response was due to expression of hCD40L.

Upregulation of immuno accessory gene in control cells by hCD40L-transfected B-CLL cells. It was previously reported that CD40L expressing MRC-5 and HeLa feeder cells could transactivate control B-CLL cells. To examine transactivity of hCD40L-transfected B-CLL cells, control B-CLL cells were first color labeled by electroporating FITC-conjugated dextran (500 kD). FITC-dextran was incorporated into 100% of the control B-CLL cells. The FITC labeled cells were then mixed with hCD40L-transfected B-CLL cells from the same donor followed by co-culturing at 37° C. for 24 hours prior to immunostaining with PE-conjugated Mab against CD86 followed by FACS analysis of the FITC positive cell population. The expression level of CD86 increased significantly on the control cells after co-culturing with the hCD40L-transfected B-CLL cells. Greater than a 4-fold increase in the mean fluorescence intensity of CD86 was observed, which was repeated with cells from 2 different donors. This demonstrated that hCD40L-transfected B-CLL cells could upregulate CD86 expression on control cells by a bystander effect.

Validation of B-CLL cell transfection procedure under cGMP guidelines. The above results demonstrated that B-CLL cells could be efficiently and consistently transfected with hCD40L DNA in regular preclinical laboratories, and the processed B-CLL cells were biologically functional. The B-CLL cell process procedure was transferred to the Center for Cell and Gene Therapy (CAGT) at Baylor College of Medicine. Five CLL patients' cell samples were processed on different dates under CAGT cGMP facility guidelines. The cell viability of the processed B-CLL cells was 82%±4%, and the hCD40L expression reached 64%±15% at 3 hours post transfection, prior to cryopreservation. By the end of the process, greater than 6 cancer vaccine doses (2e7 cells/vial) were frozen down for each patient. Up to 10 doses were cryopreserved for some patients depending on the starting cell number, which ranged from 1e8 to 5e8.

The whole process from thawing patients' B-CLL cells to cryopreservation of the vaccines took approximately 7 hours, which suggests this rapid non-viral gene modification technology is suitable for vaccine production under cGMP guidelines.

Phase I/II Human Study. In the phase I/II study design patients were administered a fixed number (20 million) of IL-2 transfected B-CLL cells and an escalated number of hCD40L transfected cells (0.2 million, 2 million, 20 million). Seven patients received vaccines. No adverse events were reported. The data are summarized in Table 3 below.

TABLE 3

| Patient | | Immunolgical | |
|---|---|---|---|
| ID | Dose Level/Status | Response | Clinical Response |
| #1 | I/Completed | ± | WBC Stable |
| #2 | I/Completed | ± | WBC Stable |
| #3 | I/Completed | ± | WBC Stable; ˜50% |
| | | | decrease in |
| | | | cervical/submental |
| | | | nodes |
| #4 | III/Completed | ± | WBC Stable |
| #5 | III/Completed | ++ | WBC Stable |
| #6 | III/Completed | ++ | WBC Stable |
| #7 | III/Completed | ± | WBC Stable |
| 3 | Withdrawn for medical | | |
| patients | reasons before vaccine | | |
| | administration | | |

### Example 4

Efficient reporter gene delivery to primary, Leukemia B cells by mRNA transfection. CLL-B cells were electroporated with 5′-end capped mRNA encoding for the marker gene, eGFP, which was obtained by in vitro transcription of the full-length cDNA (on pCI backbone) with a commercially available T7 polymerase kit (Ambion). The transfected cells were analyzed by FACS for transfection efficiency measuring eGFP expression and for cell viability by PI exclusion. Data showed that both cell viability and Transfection efficiency were 90% at 3 hours post-transfection.

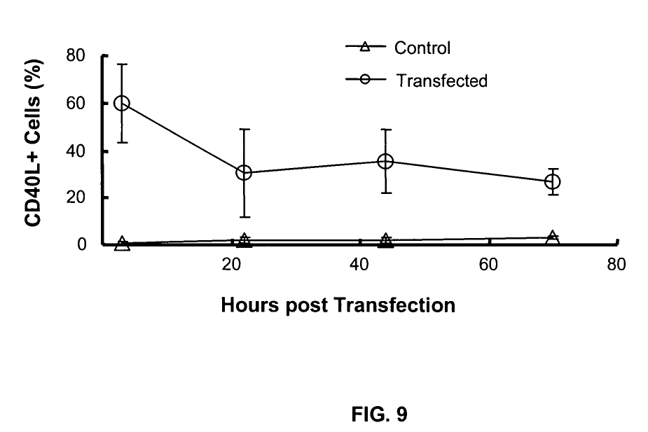

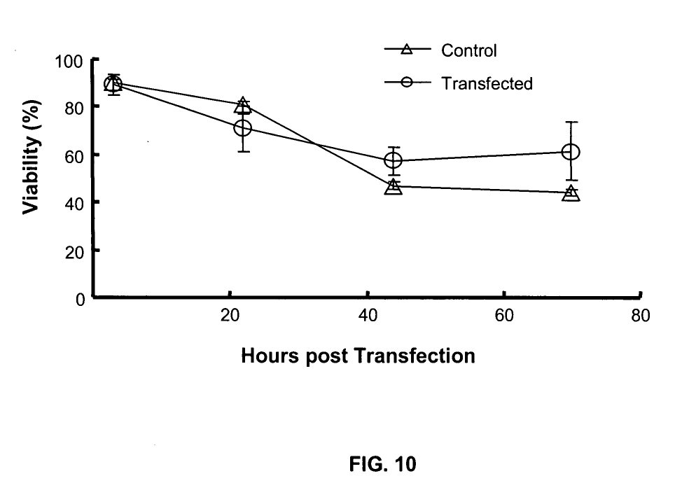

Efficient hCD40L gene delivery to primary, Leukemia B cells by mRNA transfection. Full-length hCD40L mRNA was obtained by the same procedure as described above for eGFP and electroporated into CLL-B cells. The transfected cells were immunostained using a FITC-conjugated monoclonal antibody to hCD40L (BD Pharmingen) and analyzed by FACS. Cell surface expression of hCD40L was achieved in greater than 50% of the CLL-B cells as early as 2 hours post-transfection and persisted for at least 72 hours (FIG. 9). Cell viability, when normalized against control cells was 90% (FIG. 10).

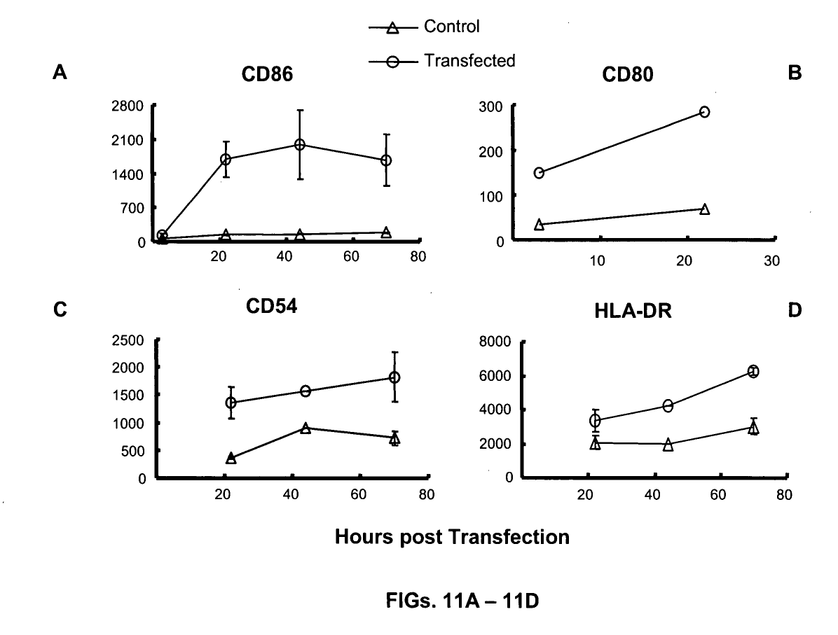

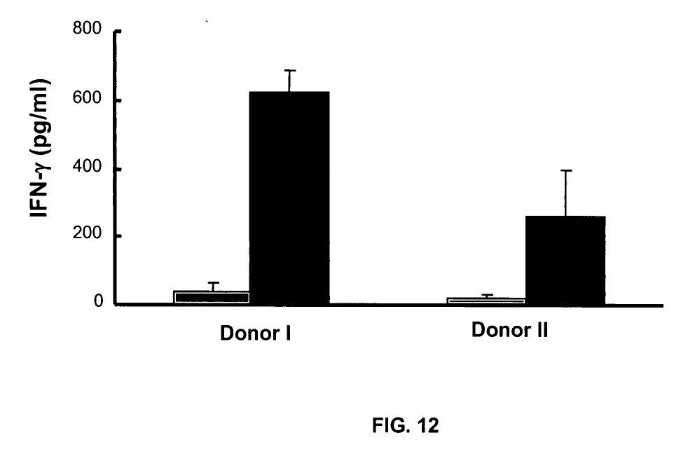

FACS analysis of the co-stimulatory molecules revealed that hCD40L expression correlated with an up-regulation of CD80, CD86, CD54, and HLA-DR (FIGS. 11A-11D). Significant up-regulation of CD86 was detected as early as 2-4 hours post transfection. The mean fluorescence intensity of CD86 expression was increased approximately 10 fold versus control cells. Control and transfected CLL-B cells were mixed and co-cultured with allo-lymphocytes 1-3 hours post transfection. IFN-γ production was measured with a commercially available ELISA kit after co-culture for 3 days. The transfected cells elicited a significantly higher level of IFN-γ production than control cells (p<0.002, sutedent t-test) (FIG. 12). These studies indicate that transient gene expression by mRNA is suitable for both in vivo and ex vivo therapies, particularly immunotherapies.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,683,202
U.S. Pat. No. 5,612,207
U.S. Pat. No. 5,720,921
U.S. Pat. No. 5,925,565
U.S. Pat. No. 5,928,906
U.S. Pat. No. 5,935,819
U.S. Pat. No. 6,074,605
U.S. Pat. No. 6,090,617
U.S. Pat. No. 6,090,617
U.S. Pat. No. 6,485,961
U.S. Pat. No. 6,617,154
U.S. Pat. No. 6,773,669
U.S. Pat. Ser. No. 10/781,440
U.S. Pat. Ser. No. 10/675,592
U.S. Pat. Ser. No. 10/225,446
U.S. Pat. Ser. No. 10/225,446
U.S. Pat. Ser. No. 10/080,272
Almendro et al., J. Immunol., 157(12):5411-5421, 1996.
Angel et al., Mol. Cell. Biol., 7:2256, 1987a.
Angel et al., Cell, 49:729, 1987b.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1996.
Banerji et al., Cell, 27(2 Pt 1):299-308, 1981.
Banerji et al., Cell, 33(3):729-740, 1983.
Berkhout et al., Cell, 59:273-282, 1989.
Biagi et al., Hum. Gen Ther., 14(6):545-559, 2003.
Blanar et al., EMBO J., 8:1139, 1989.
Bodine and Ley, EMBO J., 6:2997, 1987.
Boshart et al., Cell, 41:521, 1985.
Bosze et al., EMBO J., 5(7):1615-1623, 1986.
Braddock et al., Cell, 58:269, 1989.
Bulla and Siddiqui, J. Virol., 62:1437, 1986.
Califano et al., Cancer Res., 56(11):2488-2492, 1996.
Campbell and Villarreal, Mol. Cell. Biol., 8:1993, 1988.
Campere and Tilghman, Genes and Dev., 3:537, 1989.
Campo et al., Nature, 303:77, 1983.
Carbonelli et al., FEMS Microbiol. Lett., 177(1):75-82, 1999.
Celander and Haseltine, J. Virology, 61:269, 1987.
Celander et al., J. Virology, 62:1314, 1988.
Chandler et al., Cell, 33:489, 1983.
Chandler et al., Proc. Natl. Acad. Sci. USA, 94(8):3596-601, 1997.
Chang et al., Mol. Cell. Biol., 9:2153, 1989.
Chatteijee et al., Proc. Natl. Acad. Sci. USA, 86:9114, 1989.
Chen et al., Oncogene, 8:2159-66, 1993.
Choi et al., Cell, 53:519, 1988.
Cocea, Biotechniques, 23(5):814-816, 1997.
Cohen et al., J. Cell. Physiol., 5:75, 1987.
Connor et al., Int. J. Gynecol. Cancer, 3(2):103-109, 1993.
Connor et al., J. Exp. Med., 177:1127-1134, 1993.
Costa et al., Mol. Cell. Biol., 8:81, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Jager et al., Semin. Nucl. Med., 23(2):165-179, 1993.
De Villiers et al., Nature, 312(5991):242-246, 1984.
Deschamps et al., Science, 230:1174-1177, 1985.
Dilloo et al., Blood, 90(5):1927-1933, 1997.
Doolittle and Ben-Zeev, Methods Mol Biol, 109:215-237, 1999.
Dranoff et al., Proc. Natl. Acad. Sci. USA, 90:3539-3543, 1993.
Edbrooke et al., Mol. Cell. Biol., 9:1908, 1989.
Edlund et al., Science, 230:912-916, 1985.
Fearon and Vogelstein, Cell, 61(5):759-767, 1990.
Feng and Holland, Nature, 334:6178, 1988.
Firak and Subramanian, Mol. Cell. Biol., 6:3667, 1986.
Foecking and Hofstetter, Gene, 45(1):101-105, 1986.
Fujita et al., Cell, 49:357, 1987.
Gabriel and Teissie, Biophys. J., 76(4):2158-2165, 1999.
Gansbacher et al., Cancer Res.,50(24):7820-7825, 1990.
Gansbacher et al., J. Exp. Med., 172:1217-1224, 1990.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodburn et al., Cell, 45:601, 1986.
Greene et al., Immunology Today, 10:272, 1989
Grosschedl and Baltimore, Cell, 41:885, 1985.
Gulbis and Galand, Hum. Pathol., 24(12):1271-1285, 1993.
Harding, Eur. J Immunol., 22(7):1865-1869, 1992.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Herr and Clarke, Cell, 45:461, 1986.
Hibino et al., Biophys. J., 64(6):1789-1800, 1993.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jain et al., Ann. Surg. Oncology, 10:810-820, 2003.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Kakorin et al., Biophys. Chem., 58(1-2):109-116, 1996.
Karin et al., Mol. Cell. Biol., 7:606, 1987.
Karp et al., J. Immunol., 150(3):896-908, 1993.
Kasaian et al., Immunity, 16:559-569, 2002.
Katinka et al., Cell, 20:393, 1980.
Kawamoto et al., Mol. Cell. Biol., 8:267, 1988.
Kiledjian et al., Mol. Cell. Biol., 8:145, 1988.
Kim et al., J. Virol., 76(4)1892-1903, 2002.
Kimura et al., Arch. Otolaryngol Head Neck Surg., 129:1181-1185, 2003.
Kishida et al., Gene Therapy, 8:1234-1240, 2001.
Kishida et al., Mol. Ther., 8(4):552-558, 2003.
Klamut et al., Mol. Cell. Biol., 10:193, 1990.
Knutson and Yee, Anal. Biochem., 164(1):44-52, 1987.
Koch et al., Mol. Cell. Biol., 9:303, 1989.
Kotnik et al., Bioenerg., 43:281-291; 45:3-16, 1998.
Kraus et al. FEBS Lett., 428(3):165-170, 1998.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Lareyre et al., J. Biol. Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Lee et al., DNA Cell Biol., 16(11):1267-1275, 1997.
Lee et al., Nature, 294:228, 1981.
Lee et al., Nucleic Acids Res., 12:4191-206, 1984.
Levenson et al., Hum. Gene Ther., 9(8):1233-1236, 1998.
Levinson et al., Nature, 295:79, 1982.
Li et al., In: Am. Soc. Gene Therapy, Abstract, 5^{th} Annual Meeting, 2002.
Li et al., J. Leukoc. Biol., 56(5):616-624, 1994.
Lin et al., Mol. Cell. Biol., 10:850, 1990.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Ma et al., J. Immunol., 171(2):608-615, 2003.
Macejak and Samow, Nature, 353:90-94, 1991.
Majors and Varmus, Proc. Natl. Acad. Sci. USA, 80:5866, 1983.
McNeall et al., Gene, 76:81, 1989.
Mellman and Steinman, Cell, 106(3):255-258, 2001.
Miksicek et al., Cell, 46:203, 1986.
Mir, Bioelectrochem., 53:1-10, 2000.
Mordacq and Linzer, Genes and Dev., 3:760, 1989.
Moreau et al., Nucl. Acids Res., 9:6047, 1981.
Muesing et al., Cell, 48:691, 1987.
Nakamura et al., In: Handbook of Experimental Immunology (4^{th} Ed.), Weir et al. (Eds.), 1:27, Blackwell Scientific Publ., Oxford, 1987.
Neumann et al., Proc. Natl. Acad. Sci. USA, 96(16):9345-9350, 1999.
Ng et al., Nuc. Acids Res., 17:601, 1989.
Nomoto et al., Gene, 236(2):259-271, 1999.
Ondek et al., EMBO J., 6:1017, 1987.
Omitz et al., Mol. Cell. Biol., 7:3466, 1987.
Palmiter et al., Nature, 300:611, 1982.
PCT Appln. WO 03/018751
PCT Appln. WO 03/018751
PCT Appln. WO 04/031353
Pech et al., Mol. Cell. Biol., 9:396, 1989.
Pelletier and Sonenberg, Nature, 334(6180):320-325, 1988.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Ripe et al., Mol. Cell. Biol., 9:2224, 1989.
Rittling et al., Nuc. Acids Res., 17:1619, 1989.
Rols and Teissie, Biophys. J., 75(3):1415-1423, 1998.
Rosen et al., Cell, 41:813, 1988.
Sakai et al., Genes and Dev., 2:1144, 1988.
Sambrook et al., In: Molecular cloning, Cold Spring Harbor Laboratory Press, Cold Spring Harbor, NY, 2001.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Strengall et al., J. Immunology, 170:5464-5469, 2003.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Thiesen et al., J. Virology, 62:614, 1988.
Treisman, Cell, 42:889, 1985.
Tronche et al., Mol. Biol. Med., 7:173, 1990.
Trudel and Constantini, Genes and Dev. 6:954, 1987.
Tsumaki et al., J. Biol. Chem., 273(36):22861-22864, 1998.
Tyndell et al., Nuc. Acids. Res., 9:6231, 1981.
Van Meirvenne et al., Cancer Gene Ther., 9(9):787-797, 2002.
van Schooten et al., Mol Med Today, 3(6):254-260, 1997.
Van Tendeloo et al., Blood, 98(1):49-56, 2001.
Vannice and Levinson, J. Virology, 62:1305, 1988.
Vasseur et al., Proc Natl. Acad. Sci. U.S.A., 77:1068, 1980.
Wang and Calame, Cell, 47:241, 1986.
Weaver and Chizmadzhev, Bioenerg., 41:135-160, 1996.
Weber et al., Cell, 36:983, 1984.
Weinberger et al. Mol. Cell. Biol., 8:988, 1984.
Weiss et al. Proc. Am. Assoc. Cancer Res., 44(5493):1094-1095, 2003.
Weiss et al. Proc. Am. Assoc. Cancer Res., 44:1094-1095, 2003.
Wendtner, Leuk. Lymphoma, 45(5):897-904, 2004.
Wierda et al. Blood, 96(9):2917-2924, 2000.
Winoto and Baltimore, Cell 59:649, 1989.
Wu et al., Biochem. Biophys. Res. Commun., 233(1):221-2266, 1997.
Yutzey et al. Mol. Cell. Biol., 9:1397, 1989.
Zhao-Emonet et al., Biochim. Biophys. Acta, 1442(2-3):109-119, 1998.
Zhou et al., J. Biol. Chem., 270(21)12665-12669, 1995.

What is claimed is:

1. A method of producing a cancer cell expressing therapeutic proteins, the method comprising:
(a) obtaining a cancer cell composition; and
(b) transfecting cancer cells of the composition by electroporation with one or more nucleic acid molecules encoding two or more therapeutic proteins;
wherein, the transfected cancer cells express the two or more therapeutic proteins.

2. The method of claim 1, wherein the nucleic acid molecules encode at least three different therapeutic proteins.

3. The method of claim 1, wherein the cancer cell composition is obtained by biopsy, resection, aspiration, venipuncture, or leukapheresis.

4. The method of claim 1, wherein the cancer cell composition is expanded in culture prior to transfection.

5. The method of claim 1, wherein the nucleic acid is DNA or RNA.

6. The method of claim 1, wherein at least one of the therapeutic proteins is a cytokine.

7. The method of claim 6, wherein the cytokine is selected from the group consisting of IL-2, IL-4, IL-5, IL-6, IL-7, IL-8, IL-12, IL-15, IL-18, IL-21, IFN-γ, TNF-α; M-CSF and GM-CSF.

8. The method claim 1, wherein the two or more therapeutic proteins are encoded by one nucleic acid molecule.

9. The method of claim 1, wherein the two or more therapeutic proteins are encoded by at least two different nucleic acid molecules.

10. The method of claim 1, wherein the two or more therapeutic proteins comprise IL-15 and IL-21.

11. The method of claim 1, wherein the two or more therapeutic proteins comprise CD40L and IL-2.

12. The method of claim 2, wherein the at least three different therapeutic proteins comprise IL-15, IL-21, and GM-CSF.

13. The method of claim 2, wherein the at least three different therapeutic proteins comprise IL-12, IL-21, and GM-CSF.

14. The method of claim 2, wherein the at least three different therapeutic proteins comprise IL-12, IL-15, and IL-21.

15. The method of claim 2, wherein the at least three different therapeutic proteins comprise IL-12, IL-15, and GM-CSF.

16. The method of claim 1 further comprising inactivating the transfected cells.

17. The method of claim 16, wherein inactivating the transfected cells comprises irradiating the transfected cells.

18. The method of claim 16, wherein inactivating the transfected cells comprises contacting the transfected cells with a cytostatic agent or a cytotoxic agent.

19. The method of claim 1, wherein the electroporation is flow electroporation.

20. A method of treating cancer in a subject, the method comprising:
(a) producing a cancer cell according to claims 1; and
(b) administering the cancer cell to the subject.

21. The method of claim 20, wherein the cancer cell is an autologous cancer cell from the subject.

22. The method of claim 20, wherein the cancer cell is an allogenic cancer cell.

23. The method of claim 20, wherein the subject is a mammal.

24. The method of claim 23, wherein the mammal is a human.

25. The method of claim 20, wherein administering the cancer cell comprises intravenous injection, intramuscular injection, intratumoral injection, subcutaneous injection, or leukapheresis.

26. The method of claim 20, wherein the cancer cell is administered to the subject at or near a tumor in the subject.

27. The method of claim 20, wherein the cancer cell is administered to the subject at a site from which a tumor has been surgically removed from the subject.
